(19) United States
(12) Patent Application Publication
(10) Pub. No.: US 2018/0028260 A1
(43) Pub. Date: Feb. 1, 2018

(54) RADIO-FREQUENCY ELECTRICAL MEMBRANE BREAKDOWN FOR THE TREATMENT OF ADIPOSE TISSUE AND REMOVAL OF UNWANTED BODY FAT

(71) Applicant: RFEMB Holdings, LLC, North Brandford, CT (US)

(72) Inventors: Gary M. Onik, Ft. Lauderdale, FL (US); James A. Miessau, Branford, CT (US); David G. Bostwick, Orlando, FL (US)

(21) Appl. No.: 15/548,908

(22) PCT Filed: Feb. 3, 2016
(86) PCT No.: PCT/US2016/016352

§ 371 Date: Aug. 4, 2017

Related U.S. Application Data
(60) Provisional application No. 62/112,047, filed on Feb. 4, 2015.

(51) Int. Cl. A61B 18/14

(52) U.S. Cl. CPC A61B 18/1477; A61B 2018/00464

(57) ABSTRACT

An imaging, guidance, planning and treatment system integrated into a single unit or assembly of components, and a method for using same, that can be safely and effectively deployed to treat and ablate unwanted masses of adipose tissue in all medical settings, including in a physician's office or in an outpatient setting. The system utilizes the novel process of Radio-Frequency Electrical Membrane Breakdown (‘EMB’* or “RFEMB”) to destroy the cellular membranes of unwanted adipose tissue without damaging sensitive anatomical structures in the treatment, area. The system preferably comprises at least one EM B treatment probe 20, at least one ultrasound scanner, at least one trackable anesthesia needle 300, and at least one controller unit for at least partially automating the treatment process.

### CROSS-REFERENCE TO RELATED APPLICATIONS

The present invention is a continuation of U.S. Provisional Patent Application Ser. No. 62/112,047, filed Feb. 4, 2015, which is a continuation-in-part of U.S. patent application Ser. No. 14/451,333, filed Aug. 4, 2014, which claims priority to U.S. Provisional Patent Application No. 61/912,172, filed Dec. 5, 2013, U.S. Provisional Patent Application No. 61/861,555, filed Aug. 2, 2013, and U.S. Provisional Patent Application No. 61/867,048, filed Aug. 17, 2013, all of which are incorporated herein by reference,

### BACKGROUND OF THE INVENTION

1. Field of the Invention

The present invention relates generally to medical devices and treatment methods, and more particularly, to a device and method of treating unwanted fat deposits using applied electric fields.

2. Background of the Invention

Body sculpting refers to the use of either surgical or non-invasive techniques to modify the appearance of the body. In general, three (3) types of patients undergo body-sculpting procedures. Patients with focal adiposity may desire body sculpting for problem areas such as the abdomen, thighs, or hips. Patients with skin laxity of the face, neck, or arms may require treatments that tighten skin and deeper layers. Patients who have both focal adiposity and skin laxity require treatment that combines skin tightening with reduction in focal adiposity,

For patients requiring substantial fat reduction, surgical lipoplasty remains a popular method for body sculpting in the United States. However, the number of lipoplasty procedures performed annually has decreased dramatically as patients look for less invasive methods of body sculpting. The total number of procedures performed declined to 198,000 in 2009 from 245,000 in 2008 (−19%) and from 350,000 in 2000 (−44%).

Lipoplasty is associated with the highest potential for significant complications, morbidity, and mortality. Mortality occurs for about 1 in 47,000 patients and is most often caused by embolism complications of anesthesia, necrotizing fasciitis, and hypovolemic shock. Ultrasound-assisted liposuction has reduced, but not eliminated, the risk of complications. Laser-assisted liposuction demonstrates only a minor incremental benefit over conventional lipoplasty, and also exposes the patient to the risk of burns and thermal injury to deeper tissue.

Noninvasive alternatives to liposuction include cryolipolysis, radiofrequency (RF) ablation, laser therapies, injection lipolysis, and low-intensity nonthermal (mechanical) focused ultrasound. High-intensity focused ultrasound (HIFU) for the thermal ablation of adipose tissue (i.e., fat), a new therapeutic option being used in Europe and Canada, is currently under review by the United States Food and Drug Administration (FDA). Each of these technologic was developed to perform body sculpting for non-obese patients requiring reduction of focal adiposity, skin tightening, or both. Surgical liposuction remains the preferred treatment for patients in need of large-volume fat reduction or the treatment of multiple areas.

Tumescent liposuction, currently the standard of care for liposuction, is an invasive surgical procedure performed in an office setting or ambulatory surgical center by a surgeon or physician named in liposuction. Tumescent liposuction involves the injection of a wetting solution containing dilute lidocaine and epinephrine into fatty tissue, which then is suctioned out through cannulas inserted through small incisions. The lidocaine allows for local anesthesia and generally eliminates the need for general anesthesia or sedation. Nonetheless, some lipoplasty procedures are performed with the patient under intravenous sedation or general anesthesia, depending on the patient's needs. Complications of tumescent liposuction include abnormal body contour, nerve damage, fibrosis, perforations, seroma, fat embolism, deep vein thrombosis, and pulmonary embolism.

Laser-assisted lipoplasty requires fiber optic delivery of laser energy to target tissues, followed by lipoplasty. Risks include effects of both laser energy and lipoplasty. Liposuction plus laser therapy has resulted in skin tightening by as much as 7.6%. However, improvements in skin tightening using laser-assisted liposuction compared with liposuction alone appear to be only slight. Moreover, skin temperatures have reached 42° C., and a report has documented deeper tissue temperatures as high as 55° C., which is hot enough to produce fat necrosis and inflammation from the bulk heating of tissue.

Thermal damage to skin is thought to occur at temperatures as low as 44° C., and skin blood flow ceases at 45° C. Therefore, clinicians must consider the potential for significant burns and deep tissue thermal injury with this treatment method, in addition to the risks of surgical liposuction. It may be difficult to guard against thermal injury because thermal monitoring equipment that relies on surface temperature measurements cannot accurately measure deeper layer heat levels.

All of the above methods, both invasive and non-invasive, suffer from the inability to preplan the procedure and carry out the plan reliably with reproducible results. In, addition, as yet, there is no technology or method that allows the safe removal of large volumes of adipose tissue as in lipoplasty, that can use heat selectively to create skin tightening, and that can be used non-invasively such as in the other body sculpting techniques.

Non-thermal ablation treatments for the removal of unwanted tissue include irreversible electroporation (IRE), which relies on the phenomenon of electroporation. With reference to FIG. 1, electroporation refers to the fact that the plasma membrane of a cell exposed to high voltage pulsed electric fields, within certain parameters, becomes temporarily permeable due to destabilization of the lipid bilayer and the formation of pores P. The cell plasma membrane consists of a lipid bilayer with a thickness t of approximately 5 nm. With reference to FIG. 2(A), the membrane acts as a non-conducting dielectric barrier forming, in essence, a capacitor. Physiological conditions produce a natural electric potential difference due to charge separation across the membrane between the inside and outside of the cell even in the absence of an applied electric field. This resting transmembrane electric potential (V′m) ranges from 40 mv for adipose cells to 85 mv for skeletal muscle cells and 90 mv cardiac muscle cells and can vary by cell size and ion concentration among other things. However, the instant inventors are not aware of the use of any non-thermal ablation techniques for the removal of adipose tissue or unwanted body fat to date.

With continued reference to FIGS. 2(B)-2(D), exposure of a cell to an externally applied electric field E induces an additional voltage V across the membrane as long as the external field is present. The induced transmembrane voltage is proportional to the strength of the external electric field and the radius of the cell. Formation of transmembrane pores P in the membrane occurs if the cumulative resting and applied transmembrane potential exceeds the threshold voltage which may typically be between 200 mV and 1 V. Poration of the membrane is reversible if the transmembrane potential does not exceed the critical value such that the pore area is small in relation to the total membrane surface. In such reversible electroporation, the cell membrane recovers after the applied field is removed and the cell remains viable. Above a critical transmembrane potential and with longer exposure times, poration becomes irreversible, leading to eventual cell death due an influx of extracellular ions resulting in loss of homeostasis and subsequent apoptosis.

Irreversible electroporation (IRE) as an ablation method grew out of the realization that the “failure” to achieve reversible electroporation could be utilized to selectively kill undesired tissue. IRE effectively kills a predictable treatment area without the drawbacks of thermal ablation methods that destroy adjacent vascular and collagen structures. Pathology after IRE of a cell does not show structural or cellular changes until 24 hours after field exposure except in certain very limited tissue types. However, in all cases, the mechanism of cellular destruction and death by IRE is apoptotic, which requires considerable time to pass. Since it would be desirable to have an adipocyte broken open immediately for physical aspiration, IRE would therefore not be useful in conjunction with other methods of fat removal such as liposuction.

During a typical IRE treatment, one to three pairs of electrodes are placed in or around the tumor. Electrical pulses carefully chosen to induce an electrical field strength above the critical transmembrane potential are delivered in groups of ten (10), usually for nine (9) cycles. Each ten-pulse cycle takes about one (1) second, and the electrodes pause briefly before starting the next cycle. As described in U.S. Pat. No. 8,048,067 to Rubinsky, et. al and U.S. patent application Ser. No. 13/332,133 by Arena, et al. which are incorporated here by reference, the field strength and pulse characteristics are chosen to provide the necessary field strength for IRE but without inducing thermal effects as with radio frequency (RF) thermal ablation.

However, the DC pulses used in currently available IRE methods and devices have characteristics that can limit their use or add risks for the patient because current methods and devices create severe muscle contraction during treatment. This is a significant disadvantage because it requires that a patient be placed and supported under general anesthesia with neuromuscular blockade in order for the procedure to be carried out, and this carries with it additional substantial inherent patient risks and costs. Moreover, since even relatively small muscular contractions can disrupt the proper placement of IRE electrodes, the efficacy of each additional pulse train used in a therapy regimen may be compromised without even being noticed during the treatment session.

What is needed is a minimally invasive ablation technology that can avoid damaging healthy tissue.

In addition, an ablation method that can be accurately targeted at previously identified unwanted masses of adipose tissue, and that spares tissue structure inside and outside of the focal treatment area, would be advantageous.

It would also be advantageous to provide a system and method for carrying out this treatment in a medical setting such as a physician's office or outpatient setting under local anesthesia, using a method that does not require general anesthesia or a neuromuscular blockade.

### SUMMARY OF THE INVENTION

It is, therefore, an object of the present invention to provide a method for the treatment of unwanted adipose tissue masses (fat) in an outpatient or doctor's office setting via tissue ablation using electrical pulses which cause immediate cell death through the mechanism of complete break down of the membrane of the adipose tissue cell.

It is another object of the present invention to provide such a treatment method that does not require the administration of general anesthesia or a neuromuscular blockade to the patient.

It is another object of the invention to provide an accurate, controllable, predictable and reproducible method to ablate deposits of adipose tissue with an accurate, mapable and predictable cosmetic result.

It is another object of this invention to provide a combined means to ablate unwanted adipose tissue, and to also remove the lipid cellular contents released by the ablation process during the ongoing therapy session, combined with the ability to apply controlled thermal energy to shrink treated legions of skin so as to achieve cosmetically superior results in a controlled and reproducible manner.

It is another object of the invention to provide a non-invasive means of treating adipose tissue beneath the skin, using one or more non-piercing electrodes placed on the skin, while leaving the surface skin cells unaffected.

The present invention is an imaging, guidance, planning and treatment system integrated into a single unit or assembly of components, and a method for using same, that can be safely, predictably and effectively deployed to treat unwanted masses of adipose tissue (fat) in all medical settings, including in a physician's office or in an outpatient setting. The system utilizes the novel process of Radio-Frequency Electrical Membrane Breakdown (“EMB” or “RFEMB”) to destroy the cellular membranes of unwanted fat tissue, without damage to the surrounding vital structures and tissue.

RFEMB is a method for destroying fat cells which fills the void of treatment options for the removal of adipose tissue and unwanted body fat described above. RFEMB uses radiofrequency pulsed energy with instant charge reversal to disrupt cellular membranes, causing the immediate release of intracellular contents without thermal changes being created. Thus, lysing of the adipocyte by RFEMB without heat generation and the subsequent removal the lysed cell materials by liposuction cannulas represents an improvement over the current art. Yet the RFEMB technology, by manipulation of pulse number, sequences and energy levels, could also provide controlled tissue heating when advantageous for skin tightening purposes.

RFEMB can also be used in a completely non-invasive method when applied though skin contact methods. In this mode, RFEMB takes advantage of the fact that the increased diameter of a cell renders it more susceptible to membrane disruption. Thus, electrodes placed OD the skin of a patient can deliver an RFEMB treatment with preferential cell lysis occurring in the subcutaneous fat layer leaving the dermis and epidermis relatively unharmed.

In addition, it has been shown that non-thermal electrical ablation methods are very predictable and can be accurately modeled prior to treatment with good correlation between the predicted zone and the subsequent zone of necrosis. Thus, using modem imaging methods that can delineate the location size and shape of a patients adipose deposits, a plan for probe placements, energy delivery and pulsing sequences can be developed and, using this plans coupled with probe placement guidance systems, a more reproducible safer treatment can be delivered.

The use of RFEMB to achieve focal ablation of unwanted tissue while preserving vital nerves, vessels and other tissue structures, among other capabilities is disclosed in U.S. patent application Ser. No. 14/451,333 and International Patent Application No. PCT/US14/68774, which are both fully incorporated herein by reference.

RFEMB is the application of an external oscillating electric field to cause vibration and flexing of the cell membrane, which results in a dramatic and immediate mechanical tearing, disintegration and/or rupturing of the cell membrane. Unlike the IRE process, in which nano-pores are created in the cell membrane but through which little or no content of the cell is released, EMB completely tears open the cell membrane such that the entire contents of the cell are expelled into the extracellular fluid, and internal components of the cell membrane itself are exposed. EMB achieves this effect by applying specifically configured electric field profiles, comprising significantly higher energy levels (as much as 100 times greater) as compared to the IRE process, to directly and completely disintegrate the cell membrane rather than to electroporate the cell membrane. Such electric field profiles are not possible using currently available IRE equipment and protocols. The inability of current IRE methods and energy protocols to deliver the energy necessary to cause EMB explains why IRE treated specimens have never shown the pathologic characteristics of EMB treated specimens, and is a critical reason why EMB had not until now been recognized as an alternative method of cell destruction.

The system according to the present invention comprises a software and hardware system, and method for using the same, for detecting and measuring a mass of unwanted fat tissue in the body of a patient, for designing an EMB treatment protocol to ablate said unwanted fat tissue mass, and for applying said EMB treatment protocol in an outpatient or doctor's office setting. The system includes an EMB pulse generator 16, one or more EMB treatment probes 20, and one or more temperature probes 22. The system further employs a software-hardware controller unit (SHCU) operatively connected to said generator 16, probes 20, and temperature probe(s) 22, along with one or more optional devices such as trackable anesthesia needles 300, endoscopic imaging scanners, ultrasound scanners, and/or other imaging devices or energy sources, and operating software for controlling the operation of each of these hardware devices.

In some embodiments the system also comprise a liposuction cannula, operatively attached to a liposuction vacuum pump and controlled by the SHCU and which is useful to remove the released intra-cellular contents of the masses of ablated fat tissue, comprised primarily of lipids, from the treatment area.

### BRIEF DESCRIPTION OF THE DRAWINGS

FIG. 1 is a diagram of a cell membrane pore.

FIG. 2 is a diagram of cell membrane pore formation by a prior art method.

FIG. 3 is a comparison of a prior art charge reversal with an instant charge reversal according to the present invention.

FIG. 4 is a square wave from instant charge reversal pulse according to the present invention.

FIG. 5 is a diagram of the forces imposed on a cell membrane as a function of electric field pulse width according to the present invention.

FIG. 6 is a diagram of a prior art failure to deliver prescribed pulses due to excess current.

FIG. 7A is a schematic diagram depicting a USS scan of a suspect tissue mass.

FIG. 7B is a schematic diagram depicting the results of a 3D Fused Image of a suspect tissue mass.

FIG. 8 is a schematic diagram depicting the target treatment area and Predicted Ablation Zone relative to a therapeutic EMB treatment probe 20 prior to delivering treatment.

FIG. 9 is a schematic diagram of a pulse generation and delivery system for application of the method of the present invention.

FIG. 10 is a diagram of the parameters of a partial pulse train according to the present invention.

FIG. 11 is a schematic diagram depicting the target treatment area and Predicted Ablation Zone relative to a therapeutic EMB treatment probe 20 at the start of treatment delivery.

FIG. 12A is a schematic diagram of a therapeutic EMB treatment probe 20 according to one embodiment of the present invention.

FIG. 12B is a composite schematic diagram (1, 2 and 3) of the therapeutic EMB treatment probe 20 of FIG. 12A showing insulating sheath 23 in various stages of retraction.

FIG. 12C is a composite schematic diagram (1 and 2) of a therapeutic EMB treatment probe 20 according to another embodiment of the present invention.

FIG. 12D is a composite schematic diagram (1 and 2) of the therapeutic EMB treatment probe 20 of FIG. 12C showing insulating sheath 23 in various stages of retraction.

FIG. 13 is a schematic diagram depicting a pad-type device 601 incorporating multiple EMB probes 20 of the needle variety.

FIG. 14 is a schematic diagram of the enhanced trackable anesthesia needle 300 according to the present invention.

FIG. 15 is a schematic diagram depicting the positioning of a therapeutic EMB treatment probe 20 according to an embodiment of present invention proximate the treatment area 2.

FIG. 16 is a schematic diagram depicting the positioning of a therapeutic EMB treatment probe 20 comprising a thermocouple 7 according to another embodiment of the present invention proximate the treatment area 2.

FIG. 17 is a schematic diagram depicting the positioning of a therapeutic EMB treatment probe 20 comprising a side port 8 for exposure of needle 9 according to another embodiment of the present invention proximate the treatment area 2.

FIG. 18 is a schematic diagram depicting the positioning of a therapeutic EMB treatment probe 20 comprising a unipolar electrode 11 according to another embodiment of the present invention proximate the treatment area 2.

FIG. 19 is a schematic diagram depicting the positioning of a therapeutic EMB treatment probe 20 comprising a side port 8 for exposure of electrode-bearing needle 17 according to another embodiment of the present invention proximate the treatment area 2.

FIG. 20 is a schematic diagram depicting the use of two therapeutic EMB treatment probes 20 for delivery of EMB treatment.

FIG. 21 is a schematic diagram of suction device 600 according to another embodiment of the present invention.

FIG. 22 is a schematic diagram of suction device 600 of FIG. 21 incorporating an ultrasound sensor.

FIG. 23 is a schematic diagram of suction device 600 of FIG. 21 incorporated into a unitary device with one or more EMB treatment probes 20.

FIG. 24 is a schematic diagram of suction device 600 of FIG. 21 incorporating an ultrasound transducer.

FIG. 25 is a schematic diagram showing one or more electrodes 3, 4 placed directly on the surface of the patient's skin for ablation of fat tissue thereunder.

FIG. 26 is a schematic diagram of the embodiment in FIG. 25 with the addition of a cooling bath.

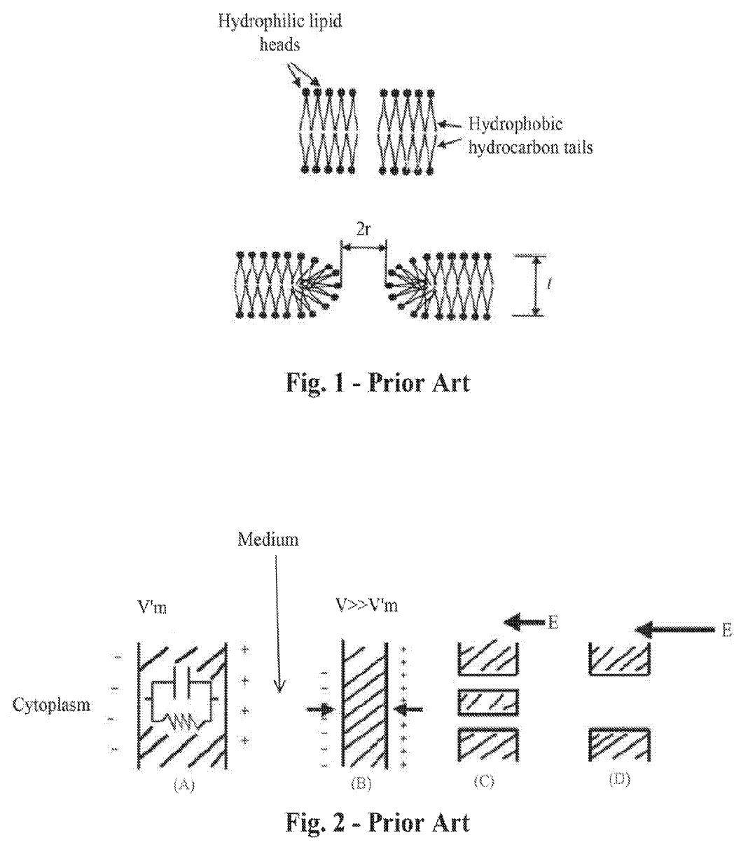

### DETAILED DESCRIPTION

In general, the software-hardware controller unit (SHCU) operating the proprietary office based adipose tissue treatment system software according to the present invention facilitates the treatment of unwanted fat tissue by directing the placement of EMB treatment probe(s) 20, and, optionally, anesthesia needle(s) 300, and by delivering electric pulses designed to cause EMB within the unwanted fat tissue to EMB treatment probe(s) 20, all while the entire process may be monitored in real time via one or more two- or three-dimensional imaging device scans taken at strategic locations to measure the extent of unwanted fat tissue cell death. In addition, the system can support the application of electrical thermal energy to support cosmetically predictable surface changes to the skin, as planned by the operator, and/or the application of liposuction treatments to remove the lipid cellular contents released by the RFEMB process during or after the RFEMB therapy session. The system is such that the treatment may be performed by a physician under the guidance of the software, or may be performed completely automatically, from the process of imaging the treatment area to the process of placing one or more probes using robotic arms operatively connected to the SHCU to the process of delivering electric pulses and monitoring the results of same. Specific components of the invention will now be described in greater detail.

EMB Pulse Generator 16

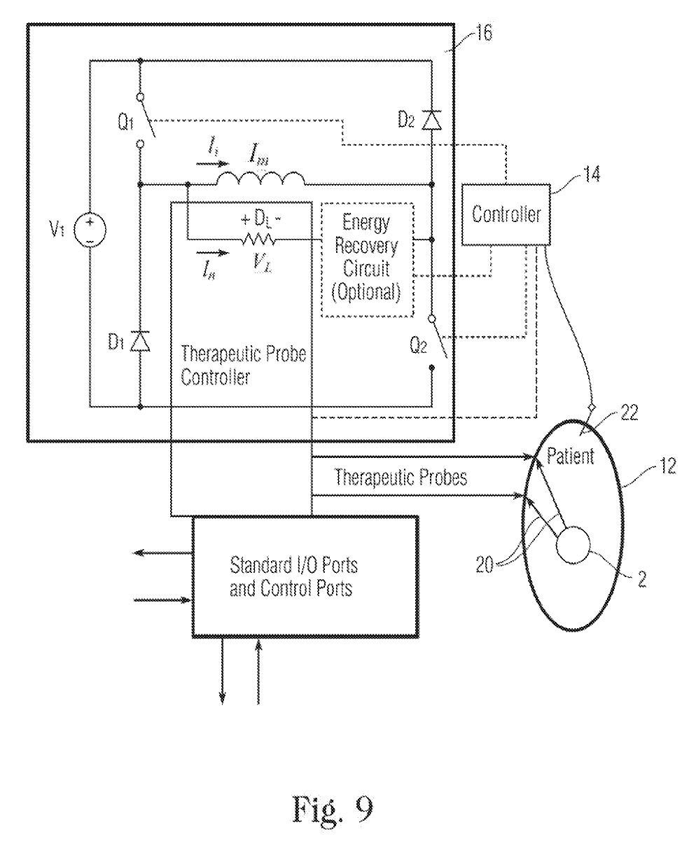

FIG. 9 is a schematic diagram of a system for generation of the electric field necessary to induce EMB of cells 2 within a patient 12. The system includes the EMB pulse generator 16 operatively coupled to Software Hardware Control Unit (SHCU) 14 for controlling generation and delivery to the EMB treatment probes 20 (two are shown) of the electrical pulses necessary to generate an appropriate electric field to achieve EMB. FIG. 9 also depicts optional onboard controller 15 which is preferably the point of interface between EMB pulse generator 16 and SHCU 14. Thus, onboard controller 15 may perform functions such as accepting triggering data from SHCU 14 for relay to pulse generator 16 and providing feedback to SHCU regarding the functioning of the pulse generator 16. The EMB treatment probes 20 (described in greater detail below are placed in proximity to the masses of unwanted fat tissue 2 which are intended to be ablated through the process of EMB and the bipolar pulses are shaped, designed and applied to achieve that result in an optimal fashion. A temperature probe 22 may be provided for percutaneous temperature measurement and feedback to the controller of the temperature at, an or near the electrodes. The controller may preferably include an onboard digital processor and a memory and may be a general purpose computer system, programmable logic controller or similar digital logic control device. The controller is preferably configured to control the signal output characteristics of the signal generation including the voltage, frequency, shape, polarity and duration of pulses as well as the total number of pulses delivered in a pulse train and the duration of the inter pulse burst interval.

With continued reference to FIG. 9, the EMB protocol calls for a series of short and intense bi-polar electric pulses delivered from the pulse generator through one or more EMB treatment probes 20 inserted directly into, or placed around the target tissue 2. The bi-polar pulses generate an oscillating electric field between the electrodes that induce a similarly rapid and oscillating buildup of transmembrane potential across the cell membrane. The built up charge applies an oscillating and flexing force to the cellular membrane which upon reaching a critical value causes rupture of the membrane and spillage of the cellular content. Bipolar pulses are more lethal than monopolar pulses because the pulsed electric field causes movement of charged molecules in the cell membrane and reversal in the orientation or polarity of the electric field causes a corresponding change in the direction of movement of the charged molecules and of the forces acting on the cell. The added stresses that are placed on the cell membrane by alternating changes in the movement of charged molecules create additional internal and external changes that cause indentations, crevasses, rifts and irregular sudden tears in the cell membrane causing more extensive, diverse and random damage, and disintegration of the cell membrane.

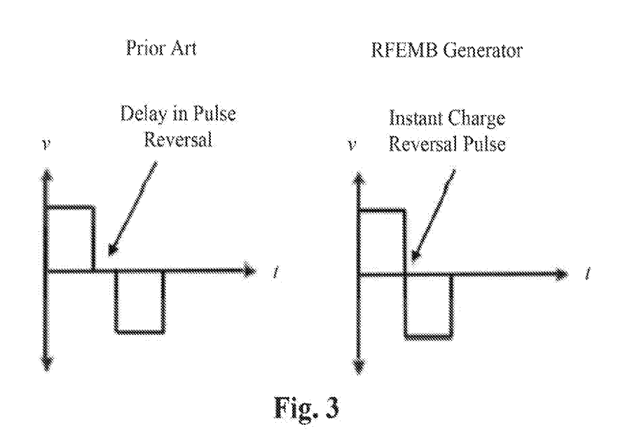

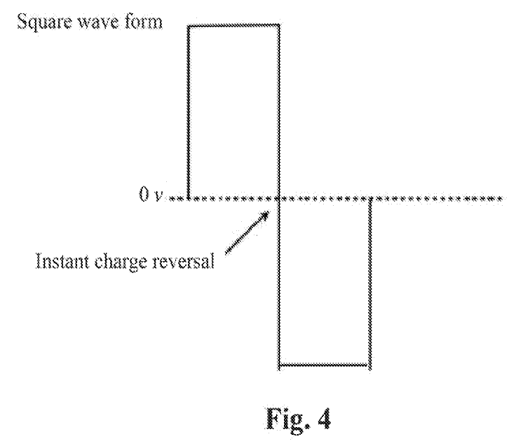

With reference to FIG. 4, in addition to being bi-polar, the preferred embodiment of electric pulses is one for which the voltage over time traces a square wave form and is characterized by instant charge reversal pulses (ICR). A square voltage wave form is one that maintains a substantially constant voltage of not less than 80% of peak voltage for the duration of the single polarity portion of the trace, except during the polarity transition. An instant charge reversal pulse is a pulse that is specifically designed to ensure that substantially no relaxation time is permitted between the positive and negative polarities of the bi-polar pulse (See FIG. 3). That is, the polarity transition happens virtually instantaneously.

The destruction of dielectric cell membranes through the process of Electrical Membrane Breakdown is significantly more effective if the applied voltage pulse can transition from a positive to a negative polarity without delay in between. Instant charge reversal prevents rearrangement of induced surface charges resulting in a short state of tension and transient mechanical forces in the cells, the effects of which are amplified by large and abrupt force reversals. Alternating stress on the target cell that causes structural fatigue is thought to reduce the critical electric field strength required for EMB. The added structural fatigue inside and along the cell membrane results in or contributes to physical changes in the structure of the cell. These physical changes and defects appear in response to the force applied with the oscillating EMB protocol and approach dielectric membrane breakdown as the membrane position shifts in response to the oscillation, up to the point of total membrane rupture and catastrophic discharge. This can be analogized to fatigue or weakening of a material caused by progressive and localized structural damage that occurs when a material is subjected to cyclic loading, such as for example a metal paper clip that is subjected to repeat bending. The nominal maximum stress values that cause such damage may be much less than the strength of the material under ordinary conditions. The effectiveness of this waveform compared to other pulse waveforms can save up to ⅕ or ⅙ of the total energy requirement.

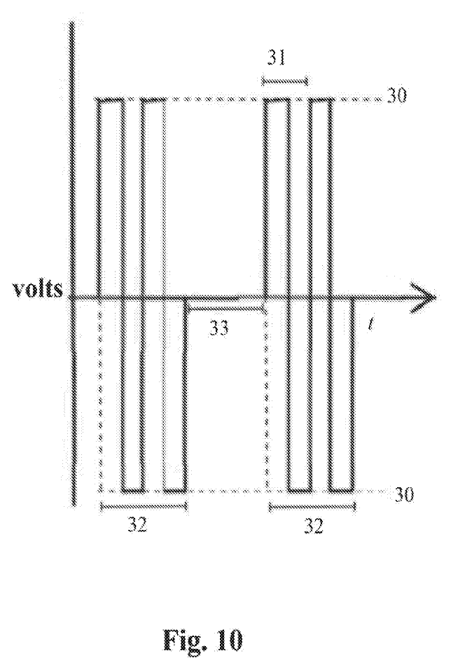

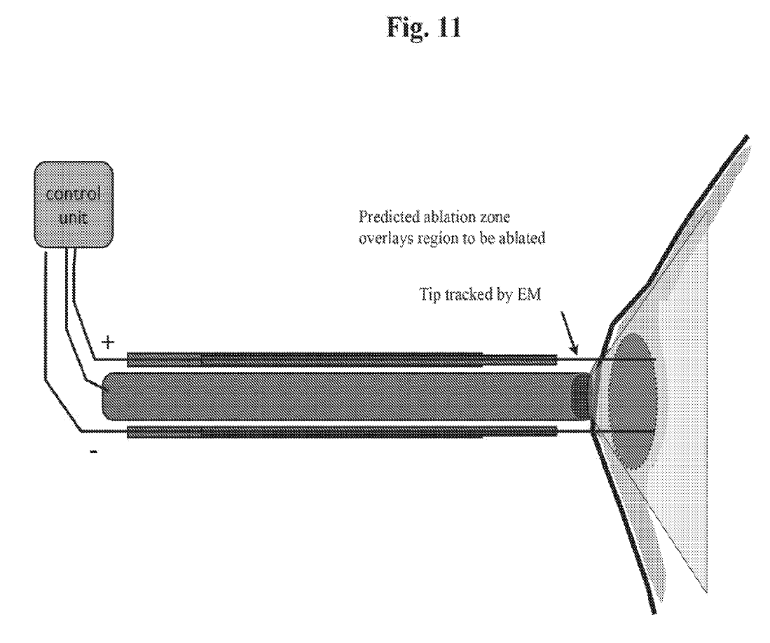

With reference to FIG. 10, another important characteristic of the applied electric field is the field strength (Volts/cm) which is a function of both the voltage 30 applied to the electrodes b the pulse generator 16 and the electrode spacing. Typical electrode spacing for a bi-polar, needle type probe might be 1 cm, while spacing between multiple needle probe electrodes can be selected by the surgeon and might typically be from 0.75 cm to 1.5 cm. A pulse generator for application of the present invention is capable of delivering up to a 10 kV potential. The actual applied field strength will vary over the course of a treatment to control circuit amperage which is the controlling factor in heat generation, and patient safety (preventing large unanticipated current flows as the tissue impedance falls during a treatment). Where voltage and thus field strength is limited by heating concerns, the duration of the treatment cycle may be extended to compensate for the diminished charge accumulation. Absent thermal considerations, a preferred field strength for EMB is in the range of 1,500 V/cm to 10,000 V/cm.

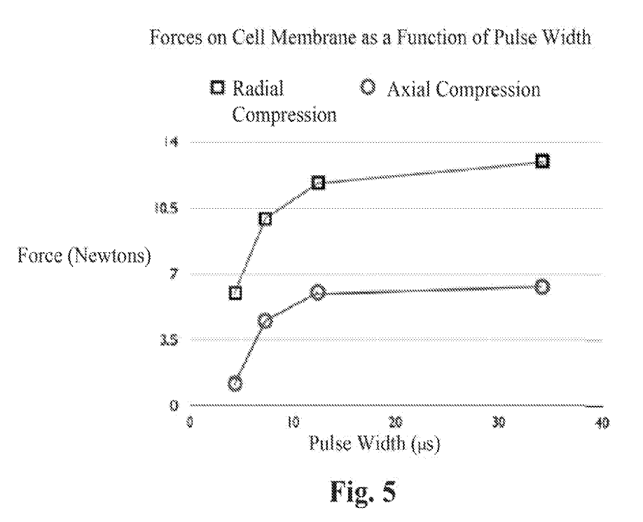

With continued reference to FIG. 10, the frequency 31 of the electric signal supplied to the EMB treatment probes 20, and thus of the field polarity oscillations of the resulting electric field, influences the total energy imparted on the subject tissue and thus the efficacy of the treatment but are less critical than other characteristics. A preferred signal frequency is from 14.2 kHz to less than 500 kHz. The lower frequency bound imparts the maximum energy per cycle below which no further incremental energy deposition is achieved. With reference to FIG. 5, the upper frequency limit is set based on the observation that above 500 kHz, the polarity oscillations are too short to develop enough motive force on the cell membrane to induce the desired cell membrane distortion and movement. More specifically, at 500 kHz the duration of a single full cycle is 2 μs of which half is of positive polarity and half negative. When the duration of a single polarity approaches 1 μs there is insufficient time for charge to accumulate and motive force to develop on the membrane. Consequently, membrane movement is reduced or eliminated and EMB does not occur. In a more preferred embodiment the signal frequency is from 100 kHz to 450 kHz. Here the lower bound is determined by a desire to avoid the need for anesthesia or neuromuscular-blocking drugs to limit or avoid the muscle contraction stimulating effects of electrical signals applied to the body. The upper bound in this more preferred embodiment is suggested by the frequency radiofrequency thermal ablation equipment already approved by the FDA, which has been deemed safe for therapeutic use in medical patients.

In addition, the energy profiles that are used to create EMB also avoid potentially serious patient risks from interference with cardiac sinus rhythm, as well as localized barotrauma, which can occur with other therapies.

EMB Treatment Probes 20

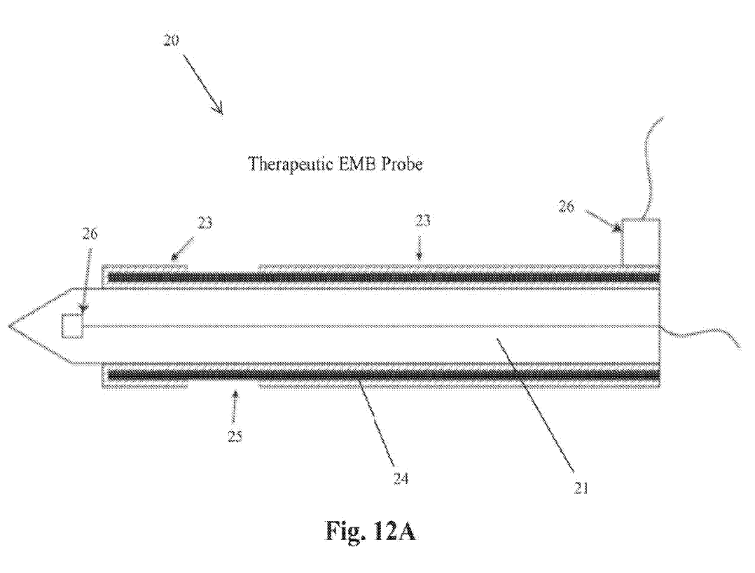

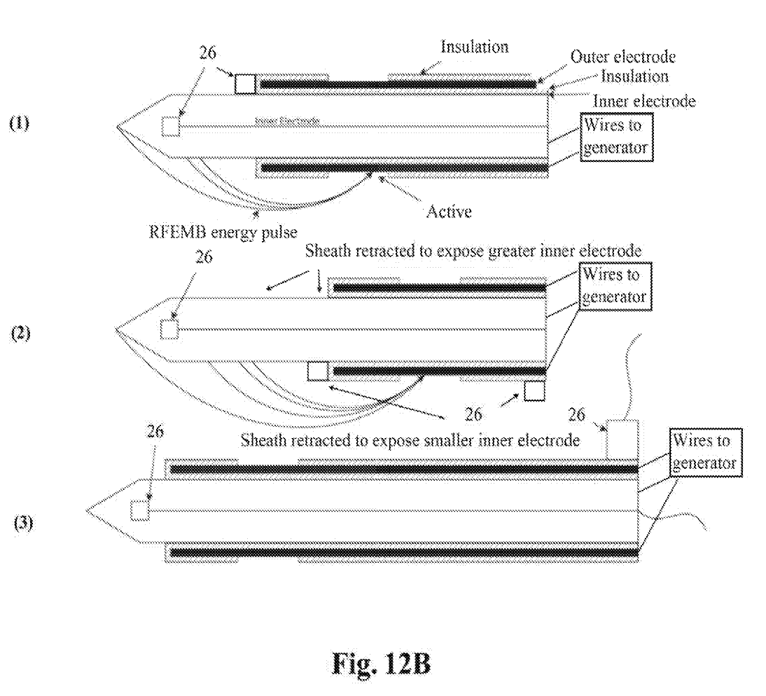

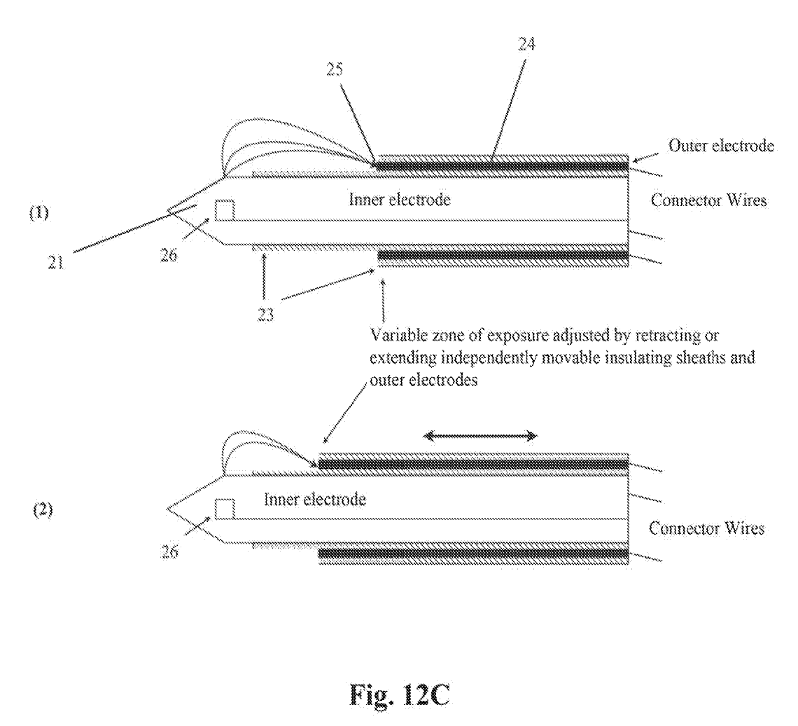

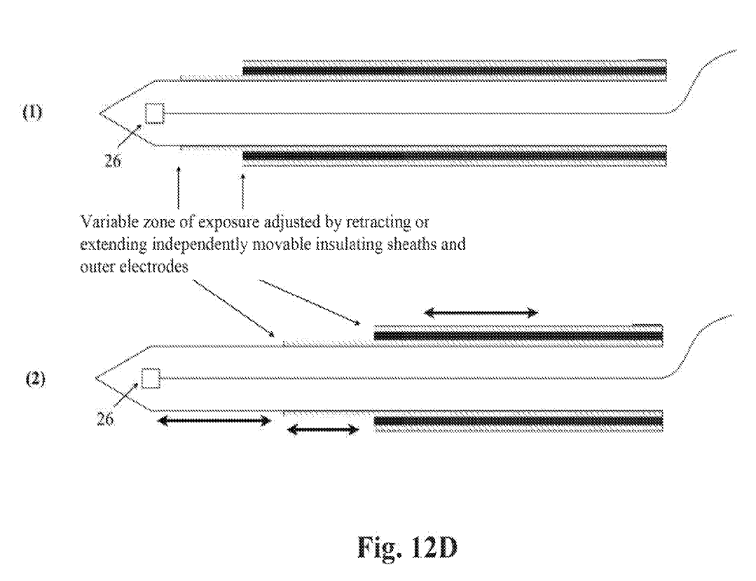

FIGS. 12A-12B depict a first embodiment of a therapeutic EMB treatment probe 20. The core (or inner electrode) 21 of EMB treatment probe 20 is preferably a needle of gage 17-22 with a length of 5-25 cm, and may be solid or hollow. Core 21 is preferably made of an electrically conductive material, such as stainless steel, and may additionally comprise one or more coatings of another conductive material, such as copper or gold, on the surface thereof. As shown in FIGS. 12A-12D, in the instant embodiment, the core 21 of treatment probe 20 has a pointed tip, wherein the pointed shape may be a 3-sided trocar point or a beveled point; however, in other embodiments, the tip may be rounded or flat. Treatment probe 20 further comprises an outer electrode 24 covering core 21 on at least one side. In a preferred embodiment, outer electrode 24 is also a cylindrical member completely surrounding the diameter of core 21. An insulating sheath 23, made of an inert material compatible with bodily tissue, such as Teflon® or Mylar®, is disposed around the exterior of core 21 and isolates core 21 from outer electrode 24. In this preferred embodiment, insulating sheath 23 is also a cylindrical body surrounding the entire diameter of core 21 and completely encapsulating outer electrode 24 except at active area 25, where outer electrode 24 is exposed directly to the treatment area 2. In an alternate embodiment, shown in FIGS. 12C-12D, insulating sheath 21 comprises two solid cylindrical sheaths wherein the outer sheath completely encapsulates the lateral area of outer electrode 24 and only the distal end of outer electrode 24 is exposed to the treatment area 2 as active area 25. Insulating sheath 23 and outer electrode 24 are preferably movable as a unit along a lateral dimension of core 21 so that the surface area of core 21 that is exposed to the treatment area 2 is adjustable, thus changing the size of the lesion created by the EMB pulses. FIGS. 12B(3) and 12C(2) depict insulating sheath 23 and outer electrode 24 advanced towards the pointed tip of core 21, defining a relatively small treatment area 2, while FIGS. 12B(2) and 12C(1) depict insulating sheath 23 and outer electrode 24 retracted to define a relatively large treatment area. Electromagnetic (EM) sensors 26 on both core 21 and it sheath 23/outer electrode 24 member send information to the Software Hardware Controller Unit (SHCU) for determining the relative positions of these two elements and thus the size of the treatment area 2, preferably in real time. EM sensors 26 may be a passive EM tracking sensor/field generator, such as the EM tracking sensor manufactured by Traxtal Inc. Alternatively, instead of utilizing EM sensors, EMB treatment probes 20 may be tracked in real time and guided using endoscopy, ultrasound or other imaging means known in the art.

One means for enabling the relative movement between core 21 and insulating sheath 23/outer electrode 24 member is to attach insulating sheath 23/outer electrode 24 member to a fixed member (i.e., a handle) at a distal end of probe 20 opposite the tip of core 21 by a screw mechanism, the turning of which would advance and retract the insulating sheath 23/outer electrode 24 member along the body of the core 21. Other means for achieving this functionality of EMB treatment probe 20 are known in the art.

One of conductive elements 21, 24 comprises a positive electrode, while the other comprises a negative electrode. Both core 21 and outer electrode 24 are connected to the EMB pulse generator 20 through insulated conductive wires, and which are capable of delivering therapeutic EMS pulsed radio frequency energy or biphasic pulsed electrical energy under sufficient conditions and with sufficient treatment parameters to achieve the destruction and disintegration of the membranes of unwanted BPH tissue, through the process of EMB, as described in more detail above. The insulated connection wires may either be contained within the interior of EMB treatment probes 20 or on the surface thereof. However, EMB treatment probes 20 may also be designed to deliver thermal radio frequency energy treatment, if desired, as a complement to or instead of EMB treatment.

In another embodiment, EMB treatment probes 20 take the form of at least one therapeutic catheter-type probe 20 for insertion into the body to treat an unwanted fat tissue mass. Catheter-type probes 20 are preferably of the flexible catheter type known in the art and having one or more central lumens to, among other things, allow probe 20 to be placed over a guide wire for ease of insertion and/or placement of probe 20 within a cavity 400 of the human body according to the Seldinger technique. A catheter for this purpose may be a Foley-type catheter, sized between 10 French to 20 French and made of silicone, latex or any other biocompatible, flexible material.

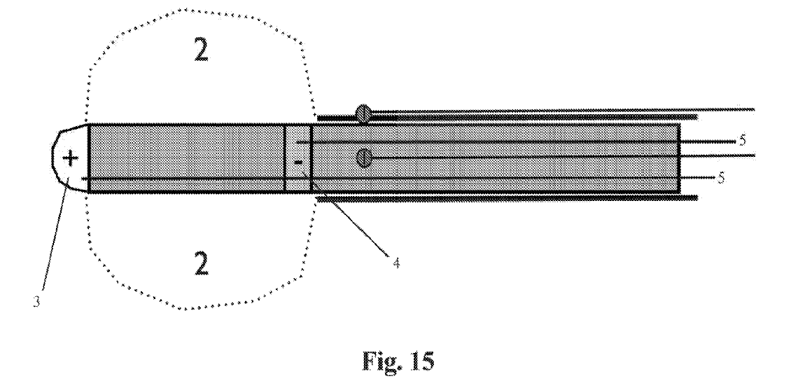

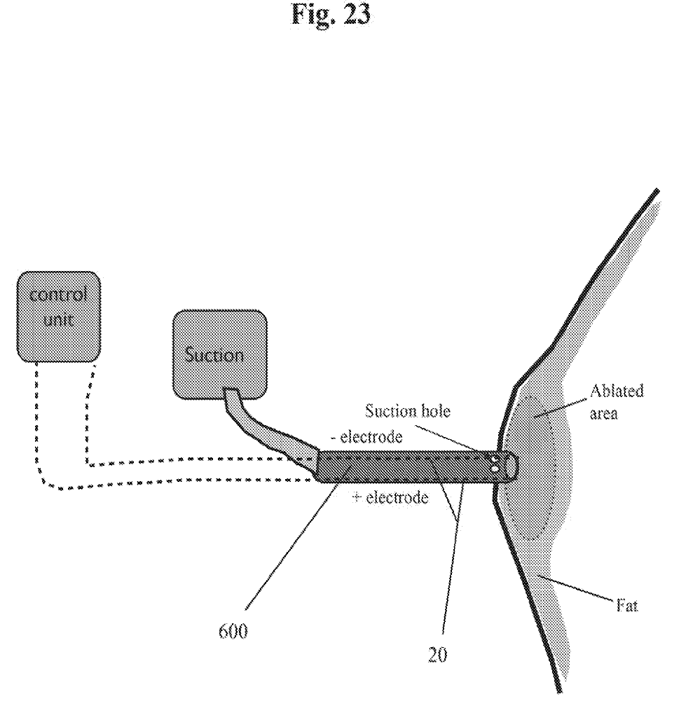

In a preferred embodiment, illustrated in FIG. 15, catheter-type probes 20 comprise one positive 3 and one negative 4 electrode disposed on an outer surface of probe 20 and spaced apart by a distance along the longitudinal axis of probe 20 such that current sufficient to deliver the EMB pulses described herein may be generated between the electrodes 3, 4. The spacing between positive 3 and negative 4 electrodes may vary by design preference, wherein a larger distance between electrodes 3, 4 provides a larger treatment area 2. FIG. 15 depicts electrodes 3, 4 on an outer surface of probe 20; alternatively, electrodes 3, 4 are integral to the surface of probe 20. In yet another embodiment, as shown in FIG. 23, one of electrodes 3, 4 (negative electrode 4 as shown in FIG. 23) may be placed on the end of an insulated sheath 23 that either partially or fully surrounds probe 20 along a radial axis thereof and is movable along a longitudinal axis of probe 20 relative to the tip thereof (on which positive electrode 3 is located as shown in FIG. 23) to provide even further customizability with respect to the distance between electrodes 3, 4 and thus the size of treatment area 2. By moving probe 20 relative to sheath 23, various distances between the electrodes can be accomplished, thus changing the size and shape of the treatment zone (see FIG. 23). Insulating sheath 23 is preferably made of an inert material compatible with bodily tissue, such as Teflon® or Mylar®. One means for enabling the relative movement between probe 20 and insulating sheath 23 is to attach insulating sheath 23 to a fixed member (i.e., a handle) at a distal end of probe 20 opposite the tip of probe 20 by a screw mechanism, the turning of which would advance and retract the insulating sheath 23 along the body of the probe 20. Other means for achieving this functionality of EMB treatment probe 20 are known in the art.

Without limitation, electrodes 3, 4 on catheter-type probes 20 may be flat (i.e., formed on only a single side of probe 20), cylindrical and surrounding probe 20 around an axis thereof, etc. Electrodes 3, 4 are made of an electrically conductive material. Electrodes 3, 4 may be operatively connected to EMB pulse generator 16 via one or more insulated wires 5 for the delivery of EMB pulses from generator 16 to the treatment area 2. Connection wires 5 may either be intraluminal to the catheter probe 20 or extra-luminal on the surface of catheter probe 20.

Electrical membrane breakdown, unlike IRE or other thermal ablation techniques, causes immediate spillage of all intracellular components of the ruptured cells into an extracellular space and exposes the internal constituent parts of the cell membrane to the extracellular space.

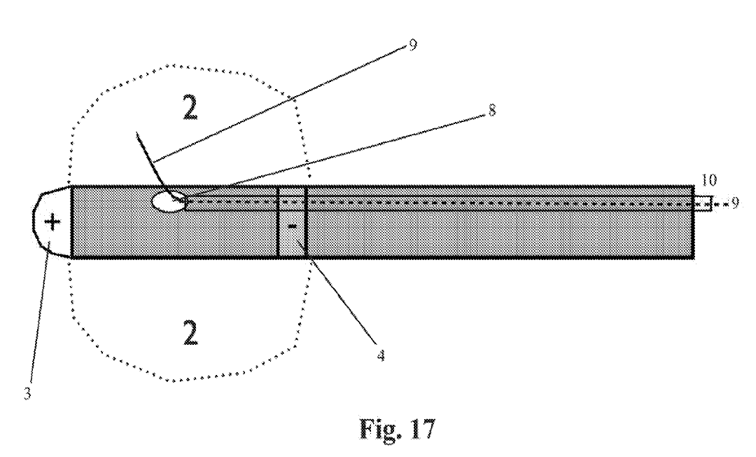

Thus, the catheter-type probe 20 according to the present invention may have a hollow interior defined by an inner lumen 10 of sufficient diameter to accommodate a spinal needle 9 of one or more standard gauges to be inserted there through for the injection of any beneficial medications or drugs into the lesion formed by EMB treatment to enhance the efficacy of said treatment (see FIG. 17). In a preferred embodiment, as shown in FIG. 17, interior lumen 10 terminates proximate an opening 8 in the side of probe 20 to allow needle 9 to exit probe 20 to access treatment area 2 for delivery of the drugs, agents, or other materials to treatment area 2. In an alternative embodiment, (not shown) interior lumen 10 may terminate, and one or more needle(s) 9 may exit, with an opening at distal end of probe 20. Alternatively, the inner lumen 10 may be sized to allow for the injection of biochemical or biophysical nano-materials there through into the EMB lesion to enhance the efficacy of the local ablative effect, or to allow injection of reparative growth stimulating drugs, chemicals or materials. A lumen 10 of the type described herein may also advantageously allow the collection and removal of tissue or intra-cellular components from the treatment area 2 or nearby vicinity, merely to remove same to aid in the healing of the treated region, or for examination or testing whether before, during or after treatment.

It will also be understood that, instead of a EMB treatment probe having a lumen 10 capable of providing a delivery path for treatment enhancing drugs, agents, or other materials, such drugs, agents or materials may be administered by any means, including without limitation, intravenously, orally or intramuscularly, and may further be injected directly into or adjacent to the target unwanted masses of fat tissue immediately before or after applying the EMB electric field.

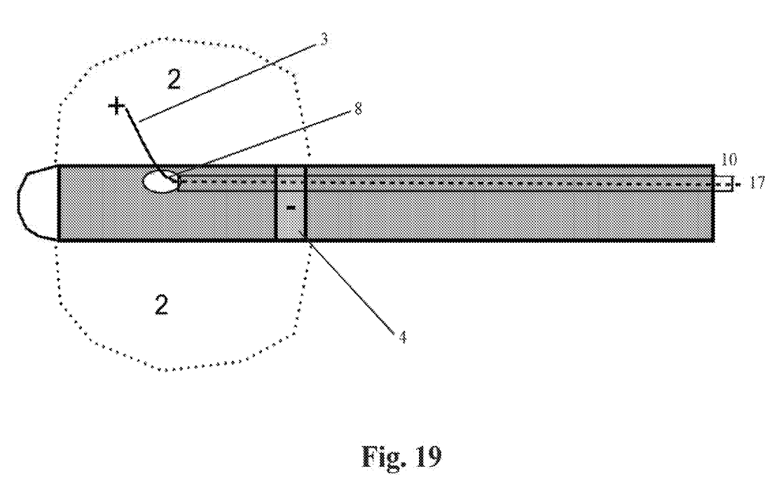

In an alternative embodiment of EMB treatment probes 20, one of either the positive (+) 3 or negative (−) 4 electrodes is on an outer surface of EMB treatment probe 20, while the other polarity of electrode is placed on the tip of a curved, electrode-bearing needle 17 inserted through lumen 10 (see FIG. 19).

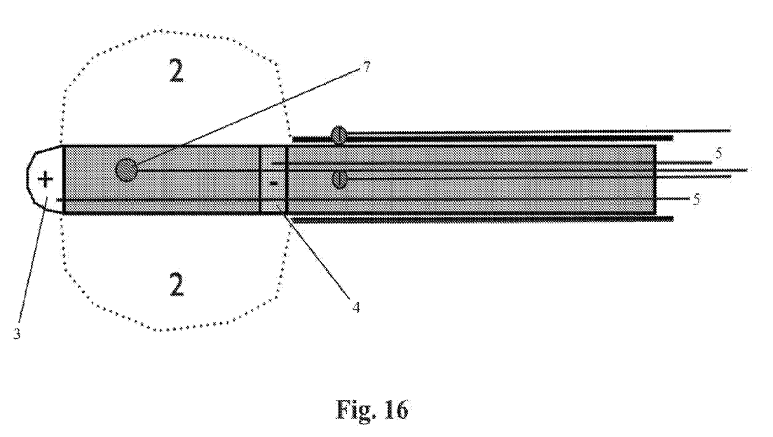

Alternatively, or in addition to the sensors described above, any of the EMB treatment probes 20 described herein may contain a thermocouple 7 (see FIG. 16), such as a Type K-40AWG thermocouple with Polyimide Primary/Nylon Bond Coat insulation and a temperature range of −40 to +180C, manufactured by Measurement Specialties. The lumen of the optional thermocouple 7 may be located on EMB treatment probe 20 such that the temperature at the tip of the probe can be monitored and the energy delivery to probe 20 modified to maintain a desired temperature at the tip of probe 20.

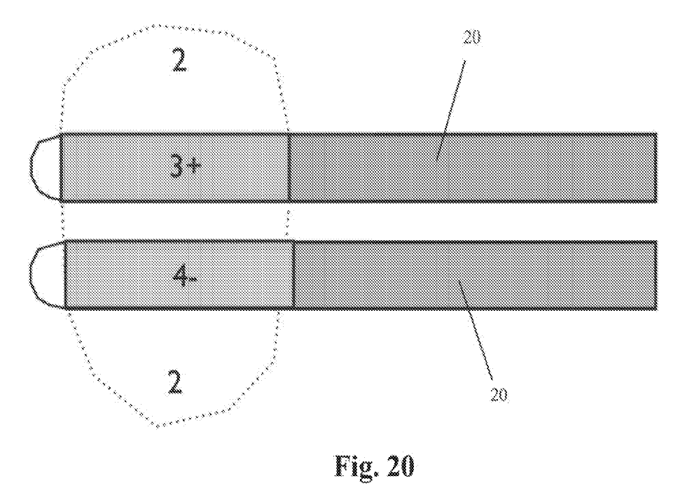

Each of the probes 20 described above also preferably comprises one or more EM sensors 26, such as those described above, on various portions of probe 20 to allow the position of the probe 20 and various parts thereof to be monitored and tracked in real time (see FIG. 20). Alternatively, instead of utilizing EM sensors, EMB treatment probes 20 may be tracked in real time and guided using endoscopy, ultrasound or other imaging means known in the art.

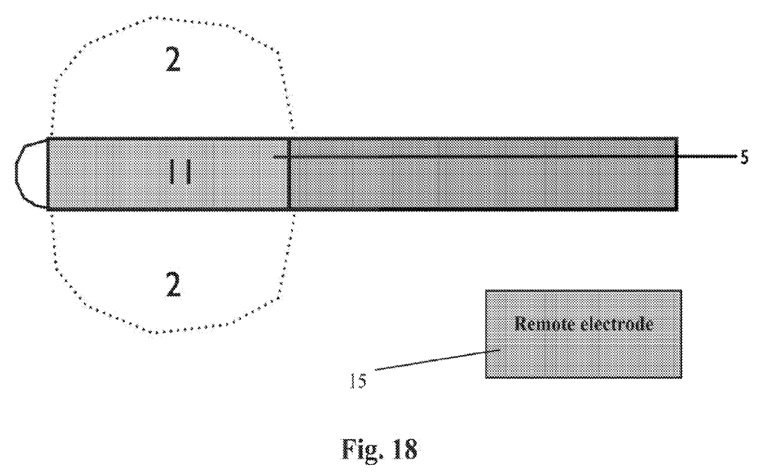

One of ordinary skill in the art will understand that the EMB treatment probe(s) 20 may take various forms provided that they are still capable of delivering EMB pulses from the EMB pulse generator 14 of the type, duration, etc. described above. For example, the EMB treatment probes 20 have been described herein as a rigid assembly, but may also be semi-rigid assembly with formable, pliable and/or deformable components. As another example, EMB treatment probes 20 may be unipolar 11 and used with an indifferent electrode placed on a remote location from the area of treatment (see FIG. 18). In yet another embodiment, two EMB treatment probes 20 may be used, wherein each probe has one each of a positive and negative electrode (See FIG. 20).

In various embodiments described herein, daring treatment of fat tissue with EMB treatment probes 20, intra-cellular contents and lipids of treated areas may be released in considerable quantity from the treated tissue. Removal of such intra-cellular contents and lipids improves the treatment outcome and results in a more efficient healing process and a more aesthetically appealing result for the patient. A combination of EMB treatment probes 20 and a separate suction device 600 may be used to achieve these benefits.

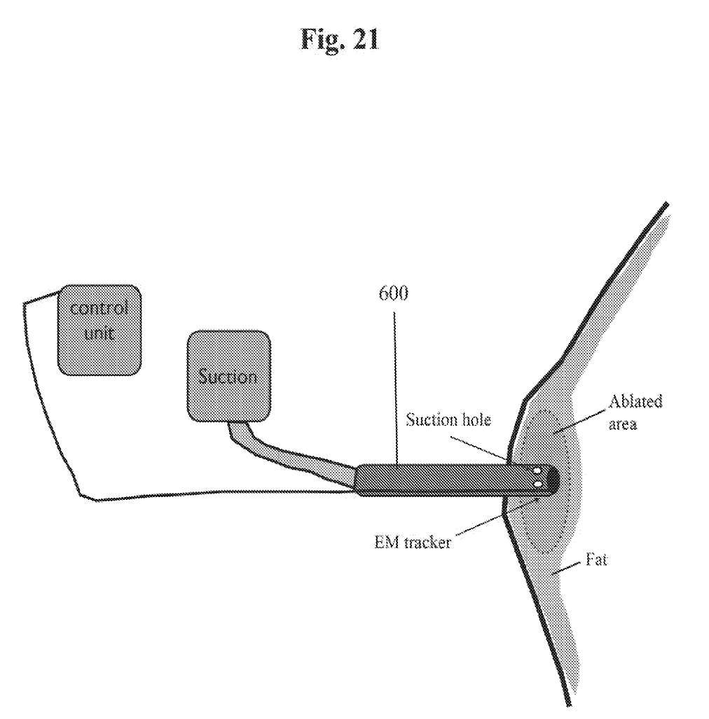

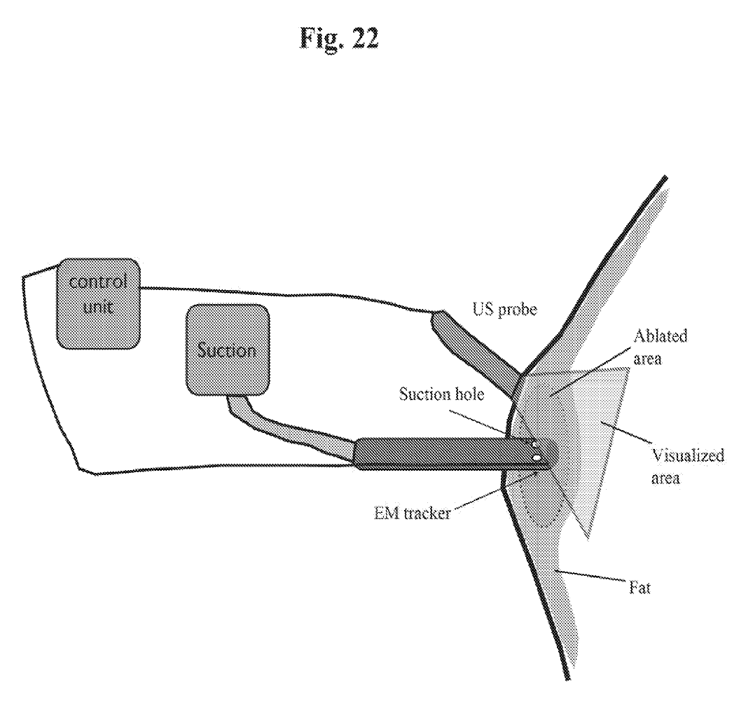

In one preferred embodiment, suction device 600 comprises a cannula with suction capability which may be separately inserted or placed into the treated area after treatment with EMB treatment probe 20 to remove the released intra-cellular contents and fat. Any type of suction device known in the art for performing liposuction or similar therapies may be used as suction device 600. Suction device 600 preferably also comprises an EM tracking device 26 and or other means for suction device 600 to be tracked by US or other surgical guidance equipment, and is operatively connected to SHCU 14. Using the 3D Fused image (described in greater detail below) the suction device 600 can be separately tracked in order to assure that the cannula is properly positioned to cover the projected area of ablation as shown by the Predicted Ablation Zone (see FIG. 21). Optionally, post-therapeutic 3D images are taken using an imaging device (MRI, CT or US), which may or may not be operatively connected to SHCU 14, and the characteristic radiographic changes of the RFEMB treatment are used to guide suction device 600 to remove the treated tissue. Alternatively, the treated tissue is removed under continuous real time ultrasound guidance (See FIG. 22).

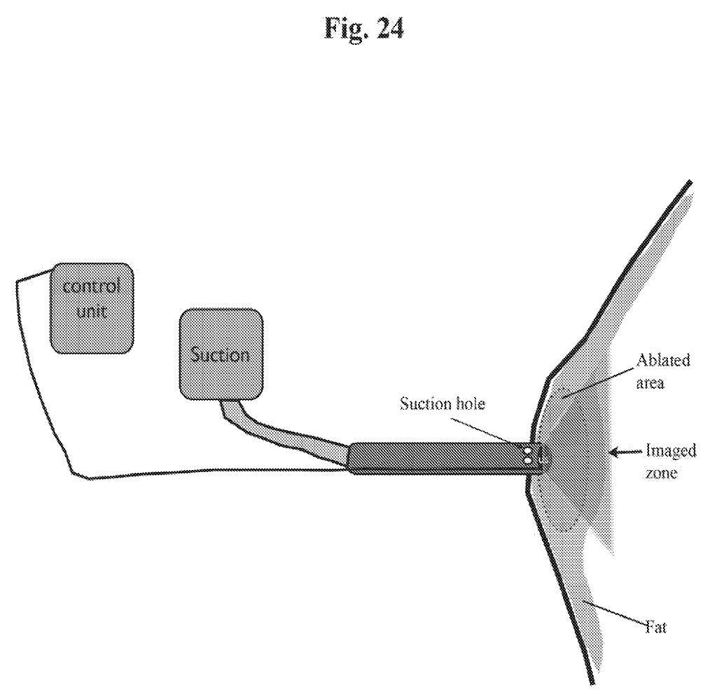

In another embodiment, therapeutic EMB probes 20 are built into suction device 600 such that treated tissue may be removed simultaneously with the delivery of EMB pulses via probe(s) 20, or in any case without removing the combined suction device 600/EMB probe 20 from the patient's body. In a preferred embodiment, the combined EMB treatment probe 20 and suction device 600 has an ultrasound transducer incorporated into its distal tip to monitor the tissue removal from inside the tissue thus improving tissue visualization (see FIG. 24).

In each of these embodiments, after tissue removal by suction device 600, the parameters of the EMB treatment can be modified, either manually by the operator or systematically by the SHCU 14 (as described below), by increasing pulse number, pulse length inter-pulse time voltage, or amplitude to provide a controlled heat treatment to the tissue to create skin tightening or hemostasis, using previously programmed or operator-determined system control parameters.

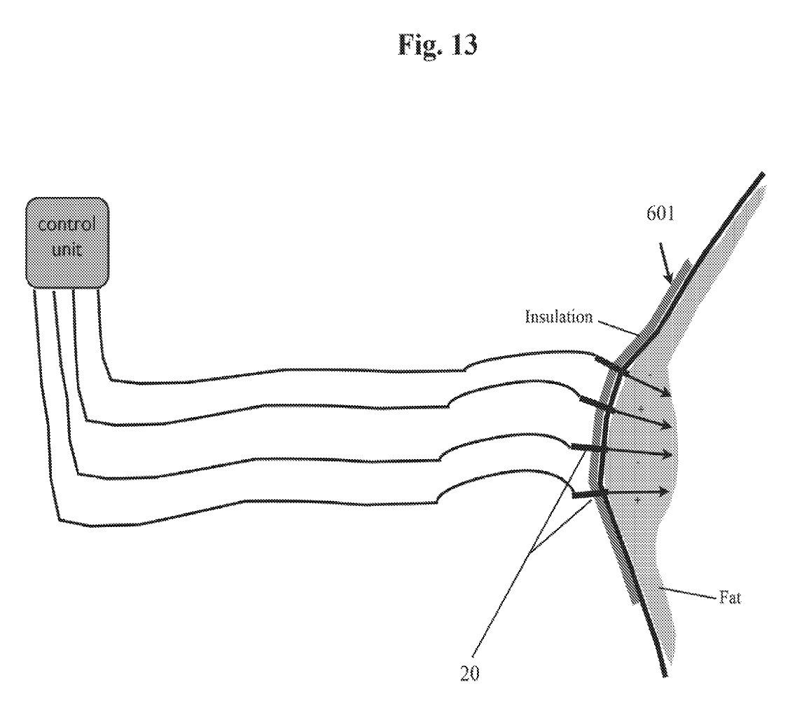

Other embodiments of EMB treatment probes 20 are designed to treat expanses of skin overlying areas of adipose tissue which is unwanted for reasons which can be purely cosmetic and/or aesthetic. Such an embodiment is shown in FIG. 13, which depicts a pad-type device 601, constructed of neoprene or another type of synthetic material, incorporating multiple EMB probes 20 of the needle variety; i.e. 22 gauge EMB treatment probes 20. Pad 601 preferably has an adhesive on one side to secure it to the patient's skin, and one more EMB probes 20 extending from the adhesive side to pierce the skin at depths that can be controlled by the physician or by SHCU 14. The layout or pattern of EMB probes 20 on pad 601 is preferably controllable as a matter of design, system or physician choice to provide the proper spacing between probes 20 and overall surface area of the treatment area 2. In such embodiments, the needle-type EMB probes 20 paired with pad 601 can each have all or any of the capabilities described herein with respect to EMB probes 20, including without limitation, EM sensor/transmitters 26 and various lengths of insulation sheathing 23 added to change the shape and extent of the treatment area 2.

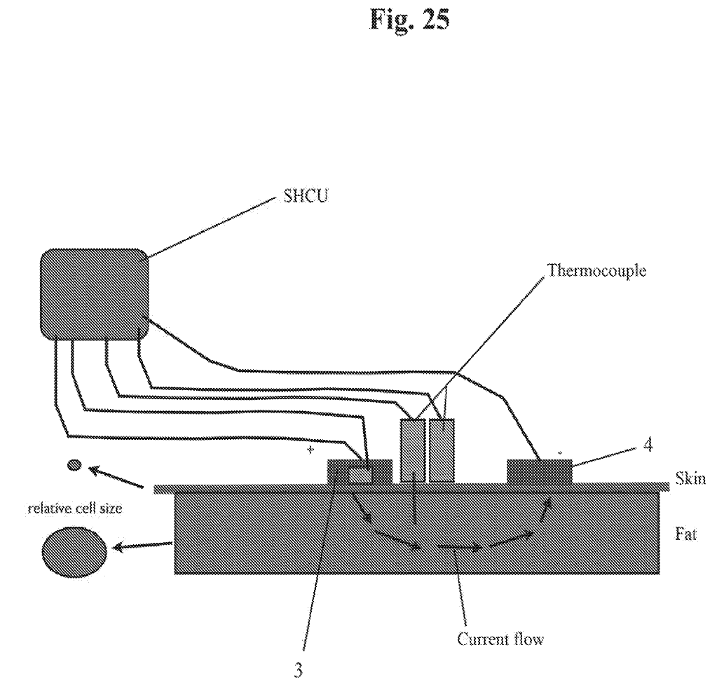

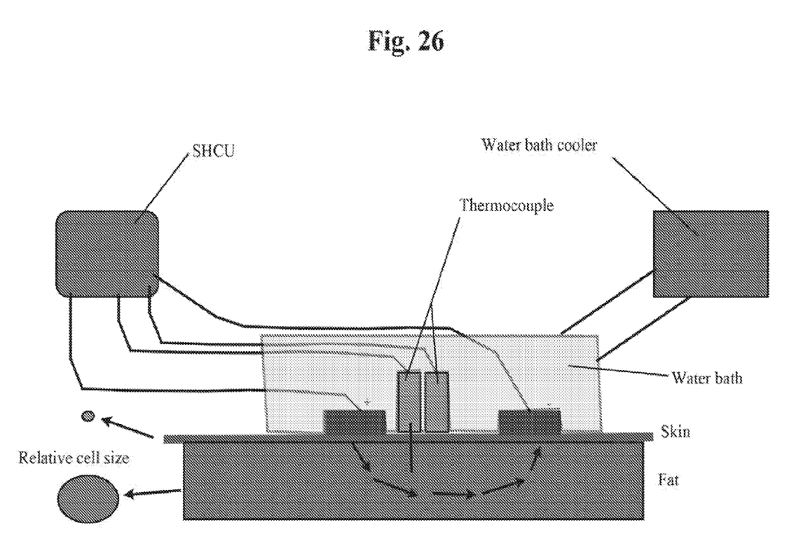

In another embodiment of the present invention, treatment of adipose tissue below the skin is accomplished non-invasively. In this embodiment, EMB treatment probes 20 are omitted in favor of one or more electrodes 3, 4 placed directly on the surface of the patient's skin. Electrodes 3, 4 are preferably configured to provide EMB pulses under the RFEMB parameters described above, as adjusted to destroy the membranes of the fat cells while leaving the skin cells unaffected (see FIG. 25). The distance between the electrodes 3, 4 may vary, as can the surface area of the electrodes 3, 4. The electrodes can be separate entities (as shown in FIG. 25) or can be incorporated into a pad (not shown) where the intervening pad areas are insulated so that the electrodes are electrically isolated from one another. Thermocouples 7 can be incorporated into the pad both in a surface configuration to monitor temperature at the skin and/or a needle configuration that monitors temperatures in the area of ablated fat. Optionally, a cooling bath or other cooling mechanism can be incorporated into the treatment pad as a further safety feature to prevent thermal damage (see FIG. 26).

It will also be understood that, instead of a EMB treatment probe having a lumen capable of providing a delivery path for treatment enhancing drugs, such drugs may be administered by any means, including without limitation, intravenously, orally or intramuscularly and may further be injected directly into or adjacent to the target unwanted masses of fat tissue immediately before or after applying the EMB electric field.

Ultrasound Scanner

Unlike irreversible electroporation, electrical membrane breakdown EMB causes immediate visually observable tissue changes which can be monitored on ultrasound to show cellular membrane destruction and immediate cell death. As a result, the method of the present invention may include the ultrasound visual evaluation of the treated target tissue to verify treatment efficacy immediately upon completion of each tissue treatment during the ongoing therapy procedure, while the patient is still in position for additional, continued or further treatment.

Additional treatment may be immediately administered via, i.e., EMB treatment probe 20, based on the information obtained from the sensors on the probe or visual determination of treatment efficacy through visual ultrasound evaluation without removing the treatment probe from the treatment area. In this preferred embodiment, an ultrasound scanner or other medical imaging device may be operatively connected to the Software Hardware Control Unit (SHCU), described in further detail below, to enable feedback from the imaging device to be relayed directly into the visualization software provided by the SHCU.

Trackable Anesthesia Needles 300

EMB, by virtue of its bipolar wave forms in the described frequency range, does not cause muscle twitching and contraction. Therefore a procedure using the same may be carried out under local anesthesia without the need for general anesthesia and neuromuscular blockade to attempt to induce paralysis during the procedure. Rather, anesthesia can be applied locally for the control of pain without the need for the deeper and riskier levels of sedation.

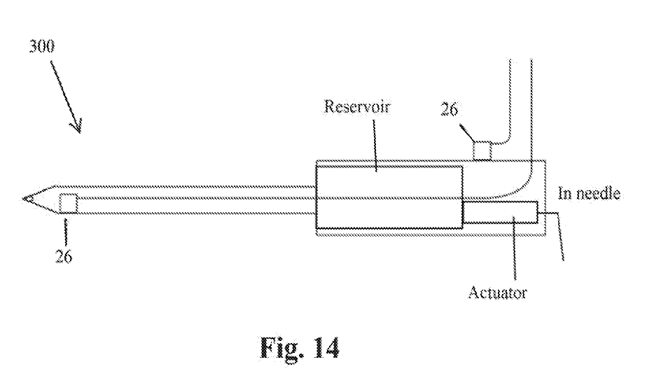

For this purpose, one or more trackable anesthesia needles 300 may be provided. With reference to FIG. 14, Anesthesia needles 300 may be of the type known in the art and capable of delivering anesthesia to potential treatment regions, including the point of entry of needle 300, EMB probe 20, or any of the other devices described herein through the skin to enhance pain relief. Anesthesia needles 300 may also comprise sensor/transmitters 26 (electromagnetic or otherwise) built into the needle and/or needle body to track the location anesthesia needle 300. Anesthesia needles 300 are preferably operatively connected to SHCU 14 to enable real-time tracking of anesthesia needle 300 by SHCU 14 and or monitor administration of anesthesia, as described in more detail below.

Alternatively, trackable anesthesia needles 300 may be omitted in favor of conventional anesthesia needles which may be applied by the physician using conventional manual targeting techniques and using the insertion point, insertion path and trajectories generated by the software according to the present invention, as described in further detail below.

Software Hardware Control Unit (SHCU) 14 and Treatment System Software

The Software Hardware Control Unit (SHCU) 14 is operatively connected to one or more (and preferably all) of the therapeutic and/or diagnostic probes/needles imaging devices and energy sources described herein: namely, in a preferred embodiment, the SHCU 14 is operatively connected to one or more EMB pulse generator(s) 16, EMB treatment probe(s) 20, and trackable anesthesia needle(s) 300 via electrical/manual connections for providing power to the connected devices as necessary and via data connections, wired or wireless, for receiving data transmitted by the various sensors attached to each connected device. SHCU 14 is preferably operatively connected to each of the devices described herein such as to enable SHCU 14 to receive all available data regarding the operation and placement of each of these devices. For example, SHCU 14 may be connected to one or more trackable anesthesia needles 300 via a fluid pump through which liquid medication is provided to anesthesia needle 300 such that SHCU 14 may monitor and/or control the volume, rate, type, etc. of medication provided through needle(s) 300.

In an alternative embodiment, SHCU 14 is also connected to one or more of the devices herein via at least one robot arm such that SHCU 14 may itself direct the placement of various aspects of the device relative to a patient, potentially enabling fully automatized and robotic treatment of certain unwanted masses of fat tissues via EMB. It is envisioned that the system disclosed herein may be customizable with respect to the level of automation, i.e. the number and scope of components of the herein disclosed method that are performed automatically at the direction of the SHCU 14. At the opposite end of the spectrum from a fully automated system, SHCU 14 may operate software to guide a physician or other operator through a video monitor, audio cues, or some other means, through the steps of the procedure based on the software's determination of the best treatment protocol, such as by directing an operator where to place the EMB treatment probe 20, etc. As examples of semi-automation, SHCU 14 may be operatively connected to at least one robotic arm comprising an alignment tool capable of supporting a treatment probe 20, or providing an axis for alignment of probe 20, such that the tip of probe 20 is positioned at the correct point and angle at the surface of the patient's skin to provide a direct path along the longitudinal axis of probe 20 to the preferred location of the tip of probe 20 within the treatment area. In another embodiment, as described in more detail below, SHCU 14 provides audio or visual cues to the operator to indicate whether the insertion path of probe 20 is correct. In each of these variations and embodiments, the system, at the direction of SHCU 14, directs the planning, validation and verification of the Predicted Ablation Zone (to be described in more detail below), to control the application of therapeutic energy to the selected region so as to assure proper treatment, to prevent damage to sensitive structures, and/or to provide tracking, storage, transmission and/or retrieval of data describing the treatment applied.

In a preferred embodiment, SHCU is a data processing system comprising at least one application server and at least one workstation comprising a monitor capable of displaying to the operator a still or video image, and at least one input device through which the operator may provide inputs to the system, i.e. via a keyboard/mouse or touch screen, which runs software programmed to control the system in two “modes” of operation, wherein each mode comprises instructions to direct the system to perform one or more novel features of the present invention. The software according to the present invention may preferably be operated from a personal computer connected to SHCU 14 via a direct, hardwire connection or via a communications network, such that remote operation of the system is possible. The two contemplated modes are Planning Mode and Treatment Mode. However, it will be understood to one of ordinary skill in the art that the software and/or operating system may be designed differently while still achieving the same purposes. In all modes, the software can create, manipulate, and display to the user via a video monitor accurate, real-time three-dimensional images of the human body, which images can be zoomed, enlarged, rotated, animated, marked, segmented and referenced by the operator via the system's data input device(s). As described above, in various embodiments of the present invention the software and SHCU 14 can partially or fully control various attached components, probes, needles or devices to automate various functions of such components, probes, needles or devices, or facilitate robotic or remote control thereof.

Planning Mode

The SHCU is preferably operatively connected to one or more external imaging sources such as an magnetic resonance imaging (MRI), ultrasound (US), electrical impedance tomography (EIT), or any other imaging device known in the art and capable of creating images of the human body. Using inputs from these external sources, the SHCU first creates one or more “3D Fused Images” of the patient's body in the region of the unwanted fat tissue. The 3D Fused Images provide a 3D map of the selected treatment area within the patient's body over which locational data obtained from the one or more probes, needles or ultrasound scans according to the present invention may be overlaid to allow the operator to plan and monitor the treatment in real-time against a visual of the actual treatment area.

In a first embodiment, a 3D Fused Image would be created from one or more MRI or CT and ultrasound image(s) of the same area of the patient's body. An MRI/CT image used for this purpose may comprise a magnetic resonance image created using, i.e., a 3.0 Telsa MRI scanner (such as Achieva, manufactured by Philips Healthcare) with a 16-channel cardiac surface coil (such as a SENSE coil, manufactured by Philips Healthcare) placed over the patient's body. MRI sequences obtained by this method preferably include: a tri-planar T2-weighted image. An ultrasound image used for this purpose may be one or more 2D images obtained from a standard biplane transrectal ultrasound probe such as the Hitachi EUB 350). The ultrasound image may be formed by, i.e., placing an EM field generator (such as that manufactured by Northern Digital Inc.) above the patient's body proximate the treatment area 2, which allows for real-time tracking of a custom ultrasound probe embedded with a passive EM tracking sensor (such as that manufactured by Traxtal, Inc.).

The 3D fused image is then formed by the software according to the present invention by encoding the ultrasound data using a position encoded data correlated to the resultant image by its fixed position to the US transducer by the US scanning device. The software according to the present invention also records of the position of the masses of fat tissue obtained as collected by ultrasound scans for later use in guiding therapy.

This protocol thus generates a baseline, diagnostic 3D Fused Image and displays the diagnostic 3D Fused Image to the operator in real time via the SHCU video monitor. Preferably, the system may request and/or receive additional 3D ultrasound images of the treatment area during treatment and fuse those subsequent images with the baseline 3D Fused image for display to the operator.

As an alternate means of creating the 3D Fused Image, a two-dimensional US sweep of the area is performed in the axial plane to render a three-dimensional ultrasound image that is then registered and fused to a previously taken MRI using landmarks common to both the ultrasound image and MRI image. Areas of adipose tissue targeted by the physician or meeting selection criteria identified in the system are identified on MRI are semi-automatically superimposed on the real-time US image. The 3D used Image as created by any one of the above methods is then stored in the non-transitive memory of the SHCU, which may employ additional software to locate and electronically tag within the 3D Fused Image specific areas, including sensitive or critical structures and areas that require anesthesia, i.e. to enable the guidance of standard or trackable anesthesia needles to those locations. The SHCU then displays the 3D Fused Image to the operator alone or overlaid with locational data from each of the additional devices described herein where available. The 3D Fused Image may be presented in real time in sector view, or the software may be programmed to provide other views based on design preference. As described above, the software may then direct the operator and/or a robotic arm to take a further ultrasound scan of the identified area of unwanted fat tissue, or in a specific location of concern based on an automated analysis of the imaging data and record the results of same, which additional imaging scan may be tracked in real time. Analysis of the image scan results which may be done by the system using automated image analysis capabilities, or a physician/technician, will indicate whether the tissue should be targeted for ablation. Thus, a 3D map of masses of targeted fat tissue in the area of concern within the patient's body may be created in this way. The software may employ an algorithm to determine where individual tissue areas should be evaluated further to ensure that all areas of concern in the region have been located evaluated, and indexed against the 3D Fused Image.

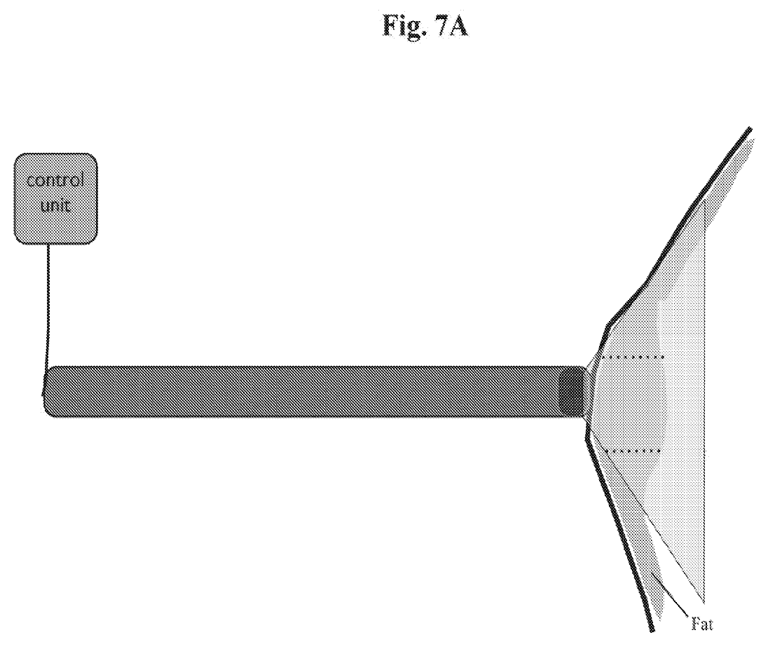

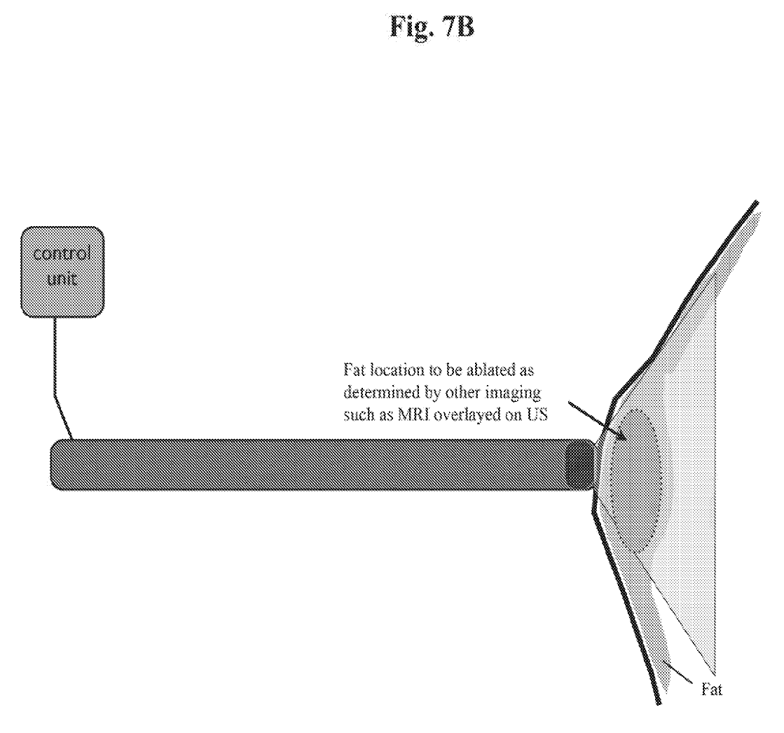

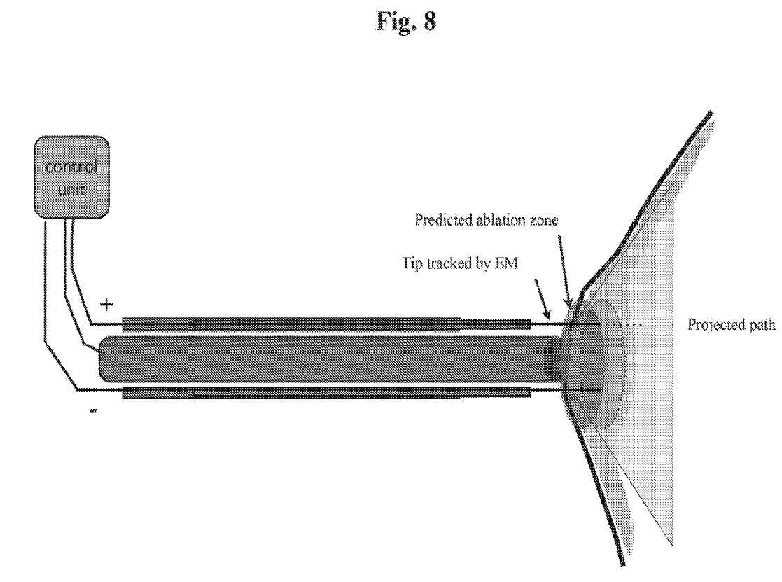

Using the image evaluation result data in conjunction with the 3D Fused Image, the software can create a targeted “3D Fused Image”, which can be used as the basis for an office based treatment procedure for the patient (see FIGS. 7A-7B). The SHCU also preferably stores the image scan information indexed to location, orientation and scan number, which information can be provided to a consulting dermatological surgeon for consultation if desired, or other treatment consultant, via a communications network to be displayed on his or her remote workstation, allowing the other treatment provider to interact with and record their findings, recommendations or analysis about each image in real time.

Upon generation of one or more 3D Fused Images of the planned treatment area and, preferably completion of the analysis of all of the image scans of the affected area, the SHCU may display to the operator via a video terminal the precise location(s) of one or more areas which require therapy, via annotations or markers on the 3D Fused Image(s); this area requiring therapy is termed the Target Treatment Zone. This information is then used by the system or by a physician to determine optimal placement of the EMB treatment probe(s) 20. Importantly, the 3D Fused Image should also contain indicia to mark the location of important anesthesia targets, which will be used to calculate a path for placement of one or more anesthesia needles for delivery of local anesthesia to the treatment area. If necessary due to changes in tissue mass size, the geographic location of each marker can be revised and repositioned, and the 3D Fused Image updated in real time by the software, using 3D ultrasound data as described above. The system may employ an algorithm for detecting changes in tissue mass size and requesting additional ultrasound scans, may request ultrasound scans on a regular basis, or the like.

In a preferred embodiment, the software may provide one or more “virtual” EMB treatment probes 20 which may be overlaid onto the 3D Fused Image by the software or by the treatment provider to determine the extent of ablation that would be accomplished with each configuration. The virtual probes also define a path to the target point by extending a line or path from the target point to a second point defining the entry point on the skin surface (or placement on the skin surface) of the patient for insertion of the real EMB treatment probe. Preferably, the software is configured to test several possible probe 20 placements and calculate the probable results of treatment to the affected area via such a probe 20 (the Predicted Ablation Zone) placement using a database of known outcomes from various EMB treatment protocols or by utilizing an algorithm which receives as inputs various treatment parameters such as pulse number, amplitude, pulse width and frequency. By comparing the outcomes of these possible probe locations to the targeted fat tissue volume as indicated by the 3D Fused Image, the system may determine the optimal probe 20 placement. Alternatively, the system may be configured to receive inputs from a physician to allow him or her to manually arrange and adjust the virtual EMB treatment probes to adequately cover the treatment area and volume based on his or her expertise. The system may utilize virtual anesthesia needles in the same way to plan treatment.

When the physician is satisfied with the Predicted Ablation Zone coverage shown on the Target Treatment Zone based on the placement and configuration of the virtual EMB treatment probes and the virtual anesthesia needles, as determined by the system or by the physician himself, the physician “confirms” in the system (i.e. “locks in”) the three-dimensional placement and energy/medication delivery configuration of the grouping of virtual EMB treatment probes and virtual anesthesia needles, and the system registers the position of each as an actual software target to be overlaid on the 3D Fused Image and used by the system for guiding the insertion of the real probe(s) and needle(s) according to the present invention (which may be done automatically by the system via robotic arms or by the physician by tracking his or her progress on the 3D Fused Image).

If necessary, EMB treatment, as described in further detail below, may be carried out immediately after the treatment planning of the patient is performed. Alternately, EMB treatment may take place days or even weeks after one or more diagnostic scanning and imaging studies are performed. In the latter case, the steps described with respect to the Planning Mode, above, may be undertaken by the software/physician at any point between diagnostic scanning and imaging and treatment.

Treatment Mode

The software displays, via the SHCU video monitor, the previously confirmed and “locked in” Target Treatment Zone, and Predicted Ablation Zone, with the location and configuration of all previously confirmed virtual probes/needles and their calculated insertion or placement points, angular 3D geometry, and optional insertion depths, which can be updated as needed at time of treatment to reflect any required changes as described above.

Using the planned locations and targets established for the delivery of anesthesia, and the displayed insertions paths, the software then guides the physician (or robotic arm) in real time to place one or more anesthesia needles and then to deliver the appropriate amount of anesthesia to the targeted locations. Deviations from the insertion path previously determined by the system in relation to the virtual needles/probes may be highlighted by the software in real time so as to allow correction of targeting at the earliest possible time in the process. This same process allows the planning and placement of local anesthesia needles as previously described. In some embodiments, the system may employ an algorithm to calculate the required amount of anesthesia based on inputs such as the mass of the tissue to be treated and individual characteristics of the patient which may be inputted to the system manually by the operator or obtained from a central patient database via a communications network, etc.

Once anesthesia has been administered, the system displays the Predicted Ablation Zone and the boundaries thereof as an overlay on the 3D Fused Image including the Target Treatment Zone and directs the physician (or robotic arm) as to the placement of each EMB treatment probe 20. The Predicted Ablation Zone may be updated and displayed in real time as the physician positions each probe 20 to give graphic verification of the boundaries of the Target Treatment Zone, allowing the physician to adjust and readjust the positioning of the Therapeutic EMB Probes, sheaths, electrode exposure and other treatment parameters (which in turn are used to update the Predicted Ablation Zone). When the physician (or, in the case of a fully automated system, the software) is confident of accurate placement of the probes, he or she may provide such an input to the system, which then directs the administration of EMB pulses via the EMB pulse generator 16 and probes 20.

The SHCU controls the pulse amplitude 30 frequency 31, polarity and shape provided by the EMB pulse generator 16, as well as the number of pulses 32 to be applied in the treatment series or pulse train, the duration of each pulse 32, and the inter pulse burst delay 33. Although only two are depicted in FIG. 10 due to space constraints, EMB ablation is preferably performed by application of a series of not less than 100 electric pulses 32 in a pulse train so as to impart the energy necessary on the target tissue 2 without developing thermal issues in any clinically significant way. The width of each individual pulse 32 is preferably from 100 to 1000 μs with an inter pulse burst interval 33 during which no voltage is applied in order to facilitate heat dissipation and avoid thermal effects. The relationship between the duration of each pulse 32 and the frequency 31 (period) determines the number of instantaneous charge reversals experienced by the cell membrane during each pulse 32. The duration of each inter pulse burst interval 33 is determined by the controller 14 based on thermal considerations. In an alternate embodiment, the system is further provided with a temperature probe 22 inserted proximal to the target tissue 2 to provide a localized temperature reading at the treatment site to the SHCU 14. The temperature probe 22 may be a separate, needle type probe having a thermocouple tip, or may be integrally formed with or deployed from one or more of the needle electrodes, or the Therapeutic EMB Probes. The system may further employ an algorithm to determine proper placement of this probe for accurate readings from same. With temperature feedback in real time, the system can modulate treatment parameters to eliminate thermal effects as desired by comparing the observed temperature with various temperature set points stored in memory. More specifically, the system can shorten or increase the duration of each pulse 32 to maintain a set temperature at the treatment site to, for example, create a heating (high temp) for the needle tract to prevent bleeding or to limit heating (low temp) to prevent any coagulative necrosis. The duration of the inter pulse burst interval can be modulated in the same manner in order to eliminate the need to stop treatment and maximizing the deposition of energy to accomplish EMB. Pulse amplitude 30 and total number of pulses in the pulse train may also be modulated for the same purpose and result.

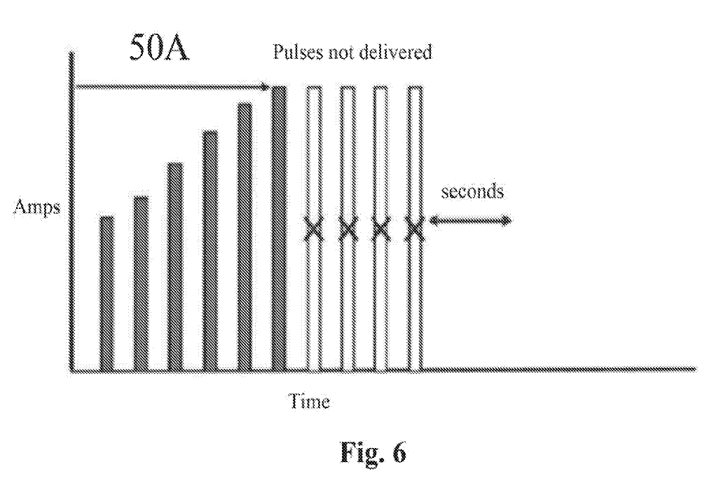

In yet another embodiment, the SHCU may monitor or determine current flow through the tissue during treatment for the purpose of avoiding overheating while yet permitting treatment to continue by reducing the applied voltage. Reduction in tissue impedance during treatment due to charge buildup and membrane rupture can cause increased current flow which engenders additional heating at the treatment site. With reference to FIG. 6, prior treatment methods have suffered from a need to cease treatment when the current exceeds a maximum allowable such that treatment goals are not met. As with direct temperature monitoring, the present invention can avoid the need to stop treatment by reducing the applied voltage and thus current through the tissue to control and prevent undesirable clinically significant thermal effects. Modulation of pulse duration and pulse burst interval duration may also be employed by the controller 14 for this purpose as described.

During treatment, the software captures all of the treatment parameters, all of the tracking data and representational data in the Predicted Ablation Zone, the Target Treatment Zone and the 3D Fused Image as updated in real time to the moment of therapeutic trigger. Based on the data received by the system during treatment, the treatment protocol may be adjusted or repeated as necessary.

The software may also store, transmit and/or forwarding treatment data to a central database located on premises in the physician's office and/or externally via a communications network so as to facilitate the permanent archiving and retrieval of all procedure related data. This will facilitate the use and review of treatment data, including for diagnostic purposes for treatment review purposes and other proper legal purposes including regulatory review.

The software may also transmit treatment data in real time to a remote proctor/trainer who can interact in real time with the treating physician and all of the images displayed on the screen, so as to insure a safe learning experience for an inexperienced treating physician, and so as to archive data useful to the training process and so as to provide system generated guidance for the treating physician. In another embodiment, the remote proctor can control robotically all functions of the system.

In other embodiments of the present invention, some or all of the treatment protocol may be completed by robotic arms, which may include an ablation probe guide which places the specially designed Therapeutic EMS Probe (or an ordinary ablation probe but with limitations imposed by its design) in the correct trajectory to the treatment area 2. Robotic arms may also be used to hold the US transducer in place and rotate it to capture images for a 3D US reconstruction. Robotic arms can be attached to an anesthesia needle guide which places the anesthesia needle in the correct trajectory to the treatment area to guide the delivery of anesthesia by the physician.

In other embodiments, the robotic arm can hold the anesthesia needle itself or a trackable anesthesia needle (see FIG. 14) with sensor-transmitters and actuators built in, that can be tracked in real time, and that can feed data to the software to assure accurate placement thereof and enable the safe, accurate and effective delivery of anesthesia to the anesthesia targets and can directly insert the needle into the targeted areas using and reacting robotically to real time positioning data supported by the 3D Fused Image and Predicted Ablation Zone data and thereby achieving full placement robotically, and upon activation of the flow actuators, the delivery of anesthesia as planned or confirmed by the physician.

In addition, the robotic arm can hold the Therapeutic EMB Probe itself and can directly insert the probe into the targeted areas of the patient using and reacting robotically to real time positioning data supported by the 3D Fused Image and Predicted Ablation Zone data and thereby achieving full placement robotically.

Robotic components capable of being used for these purposes include the Maxio robot manufactured by Perfint. In such embodiments, the software supports industry standard robotic control and programming languages such as RAIL, AML, VAL, AL, RPL, PYRO, Robotic Toolbox for MATLAB and OPRoS as well as other robot manufacturer's proprietary languages.

The SHCU can fully support Interactive Automated Robotic Control through a proprietary process for image sub-segmentation of tissue structures for planning and performing robotically guided therapeutic interventions in an office based setting.

Sub-segmentation is the process of capturing and storing precise image detail of the location size and placement geometry of the described object so as to be able to define, track, manipulate and display the object and particularly its three-dimensional boundaries and accurate location in the body relative to the rest of the objects in the field and to the anatomical registration of the patient in the system so as to enable accurate three-dimensional targeting of the object or any part thereof, as well as the three-dimensional location of its boundaries in relation to the locations of all other subsegmented objects and computed software targets and needle and probe pathways. The software sub-segments out various substructures in the treatment region in a systematic and programmatically supported and required fashion, which is purposefully designed to provide and enable the component capabilities of the software as described herein.

Having now fully set forth the preferred embodiment and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth herein.

### STATEMENT OF INDUSTRIAL APPLICABILITY

The presence of excess or unwanted adipose tissue (i.e., body fat) is a common problem for many people. Patients with focal adiposity may desire body sculpting for problem areas such as the abdomen, thighs, or hips, while patients with skin laxity of the face, neck, or arms may require treatments that tighten skin and deeper layers. The known treatments for the removal of unwanted adipose tissue have risks including the requirement to place the patient under general anesthesia, pain, disfigurement, and/or lack of effectiveness. There would be great industrial applicability in an effective ablation of adipose tissue that was minimally invasive and less traumatic than classic methods of removing such tissue by surgical excision, liposuction or other currently available means, and which could be conducted without the need for general anesthesia. The instant invention fulfills this need by utilizing Radio-Frequency Electrical Membrane Breakdown to destroy the cellular membranes of unwanted adipose tissue without denaturing the intra-cellular contents of the cells comprising the tissue, and by doing so in a focused and predictable manner under ultrasound or other imaging guidance.

We claim:

1. A method of ablating undesirable soft tissue in a living subject using radio frequency electrical membrane breakdown, the method comprising:
identifying a location of said soft tissue within said subject;
introducing at least one electrode to said location within said subject; and
applying to said soft tissue at said location, via said at least one electrode, an electric field sufficient to cause electrical membrane breakdown of a cell membrane of a plurality of cells of said soft tissue to cause immediate spillage of all intracellular components into an extracellular space and exposure of an internal constituent part of said cell membrane to said extracellular space;
wherein said undesirable soft tissue comprises adipose tissue of said living subject.

2. The method of claim 1, wherein said method is performed in an outpatient or doctor's office setting.

3. The method of claim 1, wherein said method does not require the administration of general anesthesia or a neuromuscular blockade to said living subject.

4. The method of claim 1, further comprising applying suction to said location, said suction resulting in the removal of at least a portion of said intracellular components of said plurality of cells in said location.

5. The method of claim 4, wherein said step of applying suction to said location occurs simultaneously with said step of applying said electric field to said soft tissue at said location.

6. The method of claim 4, wherein said step of applying suction to said location occurs after said step of applying said electric field to said soft tissue at said location.

7. The method of claim 6, further comprising, after said step of applying suction to said location, repeating said step of applying said electric field to said soft tissue at said location.

8. The method of claim 1, further comprising applying thermal energy at a second location on a surface of said living subject, said step of applying thermal energy at said second location resulting in the shrinkage of skin of said living subject at said second location.

9. The method of claim 1, wherein said step of applying said electric field to said soft tissue at said location is non-invasive.

10. The method of claim 1, wherein said method is monitored in real time by an ultrasound device.

11. The method of claim 1, wherein said undesirable soft tissue is located within therapeutic reach of an anatomically accessible structure of said living subject, and wherein said step of introducing said at least one electrode comprises:
inserting a cannula through said anatomically accessible structure; and
inserting one or more therapeutic EMB probes through a lumen of said cannula, wherein said one or more therapeutic EMB probes each contain one or more of said at least one electrodes.

12. A system for ablating undesirable masses of adipose tissue in a living subject using radio frequency electrical membrane breakdown, comprising:
an electric pulse generator;
at least one therapeutic probe comprising at least one electrode operatively connected to said pulse generator, said probe and pulse generator configured to apply to said adipose tissue an electric field sufficient to cause electrical membrane breakdown of a cell membrane of a plurality of cells of said soft tissue to cause immediate spillage of all intracellular components into an extracellular space and exposure of an internal constituent part of said cell membrane to said extracellular space;
an image guidance device; and
a controller operatively connected to said electric pulse generator, said therapeutic probe and said ultrasound scanner.

11. The system of claim 12, wherein said therapeutic probe comprises:
a core comprised of an electrically conductive material;
an outer electrode covering said core on at least one side; and
an insulating sheath comprised of a non-electrically-conductive material, said insulating sheath forming a barrier between said core and said outer electrode.

14. The system of claim 12, further comprising:
a pad, said pad having two sides each having a length and a width and separated by a thickness, wherein said thickness is smaller than both said length and said width, said pad further comprising an adhesive compound on one of said two sides;
wherein said at least one therapeutic probe extends out from said side of said pad comprising said adhesive compound.

15. The system of claim 12, wherein said at least one therapeutic probe comprises a suction device.

16. The system of claim 15, wherein said imaging device is an ultrasound scanner, and further comprising an ultrasound transducer incorporated into said at least one therapeutic probe.

17. The system of claim 12, wherein said image guidance device is an ultrasound scanner, and wherein said ultrasound scanner is operatively connected to said electric pulse generator.

18. The system of claim 12, wherein said image guidance device is a CT scanner, and wherein said CT scanner is operatively connected to said electric pulse generator.

19. The system of claim 12, wherein said image guidance device is an MRI imaging device, and wherein said MRI imaging device is operatively connected to said electric pulse generator.

20. The system of claim 12, wherein said at least one therapeutic probe further comprises at least one integrated electromagnetic sensor.

21. A system for ablating undesirable masses of adipose tissue in a living subject using radio frequency electrical membrane breakdown, comprising:
an electric pulse generator;
at least one therapeutic electrode operatively connected to said pulse generator, said probe and pulse generator configured to apply to said adipose tissue an electric field sufficient to cause electrical membrane breakdown of a cell membrane of a plurality of cells of said soft tissue to cause immediate spillage of all intracellular components into an extracellular space and exposure of an internal constituent part of said cell membrane to said extracellular space;
an image guidance device; and
a controller operatively connected to said electric pulse generator, said therapeutic probe and said ultrasound scanner.

22. The system of claim 21, wherein said at least one therapeutic electrode comprises one or more pads capable of being placed directly on a surface of skin of said living subject.

23. The system of claim 22, wherein said at least one therapeutic electrode comprises two or more electrodes incorporated into said pad.

24. The system of claim 22, wherein said at least one electrode comprises two or more of said pads, each of said two or more pads incorporating at least one electrode.

25. The system of claim 24, wherein said two or more pads are designed to be placed on two or more remote locations on said surface of said skin of said living subject such that said two or more pads are electrically isolated.

26. The system of claim 21, further comprising a cooling mechanism.

27. The system of claim 21, further comprising at least one thermocouple.
